(19) United States
(12) Patent Application Publication
(10) Pub. No.: US 2018/0296679 A1
(43) Pub. Date: Oct. 18, 2018

(54) PHARMACEUTICAL COMPOSITIONS OF POLYANIONIC AND NON-IONIC CYCLODEXTRIN-BASED DENDRIMERS AND USES THEREOF

(71) Applicants: Chang-Chun LING, Calgary (CA); Ping ZHANG, Calgary (CA); Aixia WANG, Calgary (CA); John KLASSEN, Edmonton (CA); Emma-Dune LERICHE, Edmonton (CA)

(72) Inventors: Chang-Chun LING, Calgary (CA); Ping ZHANG, Calgary (CA); Aixia WANG, Calgary (CA); John KLASSEN, Edmonton (CA); Emma-Dune LERICHE, Edmonton (CA)

(21) Appl. No.: 15/564,981

(22) PCT Filed: Dec. 2, 2015
(86) PCT No.: PCT/CA2015/051263

§ 371 Date: Oct. 6, 2017

Related U.S. Application Data
(60) Provisional application No. 62/144,633, filed on Apr. 8, 2015.
(60) Provisional application No. 62/237,881, filed on Oct. 6, 2015.

(51) Int. Cl. A61K 47/40; A61K 47/61; A61K 31/724; C08B 37/16

(52) U.S. Cl. CPC A61K 47/40; A61K 47/61; G01R 33/46; C08B 37/0012; A61K 31/724

(57) ABSTRACT

The present application provides pharmaceutical compositions comprising polyanionic and polynon-ionic cyclodextrin-based dendrimers. The compositions can be used as excipients, or to bind to compounds such as in the use as a rescue medicine to remove undesired drugs and metabolites from a subject Methods of use in treating a subject are also provided.

### FIELD

The present application pertains to the field of cyclodextrins. More particularly, the present application relates to cyclodextrin-based polyanionic and non-ionic dendrimers for use in pharmaceutical applications, such as excipients or rescue medicines, for example.

### BACKGROUND

Cyclodextrins (CDs) are a class of non-toxic, water-soluble D-glucose based macrocycles with a hydrophobic cavity. CDs typically vary by the number of glucose units. Common members include α-CD (6 glucose units), β-CD (7 glucose units) and γ-CD (8 glucose units), with increasing cavity size. The varying cavity sizes offer increased utility in a wide variety of applications, particularly in drug delivery models. For example, CDs can be used to form “inclusion complexes” in which a drug is included and carried within the cavity. This can be used as a pharmaceutical excipient to improve drug water solubility, chemical stability, and removal of certain drug side effects (such as undesirable taste). CDs have also drawn interest in the cosmetic and food additives industries, in the design of artificial enzymes, gene delivery vehicles, sensors and novel supramolecular assemblies.

CDs can be native or chemically modified on either or both of their primary and/or secondary faces. Typically, an inclusion complex often has lower water solubility than native CDs. Chemical modifications of CDs can change their physico-chemical properties. For example, adding a tosyl group on the primary face of the β-CD renders the molecule near insoluble at room temperature, while adding methyl groups at OH-6 and OH-2 positions significantly increases water solubility. The toxicity of the molecule can also be changed. Therefore, modification of the CD molecule may present certain advantages. However, chemical modification of CDs is typically difficult to achieve, often leading to the formation of a mixture of products that are difficult to separate.

The groups added to the primary or second face can be neutral or charged. For example, Captisol® is an excipient for use with a number of drugs. It is a polyanionic mixture of β-CD derivative having from 1 to 10 sodium sulfobutyl ether groups directly attached via oxygen atoms of the D-glucose thereto (U.S. Pat. No. 5,134,127 (Stella et al)). Capitsol is prepared by reacting a β-CD with 1,4-butyl sultone and sodium hydroxide in water. The obtained product is a mixture containing many positional and regioisomers with varying degrees of substitution at different oxygen positions on the CD, such as substitution at O-2, O-3 and O-6 on the CD. (Luna, et al., Carbohydr. Res., 299, 103-110, 1997; Luna, et al., Carbohydr. Res., 299, 111-118, 1997; Rogmann et al., Carbohydr. Res., 327, 275-285, 2000; http://www.captisol.com/faq/solution-and-solid-state-characteristics-in-captisol).

There are certain disadvantages with Captisol. As it comprises a mixture of compounds, thus resulting in varied compositions, it is difficult if not impossible to define and characterize the product compositions.

Another polyanionic CD compound currently on the market is Sugammadex (by Merck), which is a polyanionic agent obtained from γ-CD. Sugammadex blocks the activity of neuromuscular agents (Yan, et al., Drugs, 2009: 69, 919-42; Calderón-Acedos, et al., Eur. J. Hosp. Pharm. 2012: 19, 248). See also U.S. Pat. No. 6,670,340 (Zhang et al.) and U.S. Pat. No. 6,949,527 (Zhang et al.).

Non-ionic CD-based compounds are also known in the art. One example includes hydroxypropyl-beta CD (HPBCD). However, this exists in a mixture of compounds, similarly resulting in varied compositions.

There is a need for pure anionic or non-ionic CD derivatives for various applications in the pharmaceutical industry.

This background information is provided for the purpose of making known information believed by the applicant to be of possible relevance to the present invention. No admission is necessarily intended, nor should be construed, that any of the preceding information constitutes prior art against the present invention.

### SUMMARY

An object of the present invention is to provide improved pure polyanionic and non-ionic cyclodextrin-based compounds for use in various pharmaceutical applications.

In accordance with an aspect of the present invention, there is provided a pharmaceutical composition comprising a polyanionic compound of the formula:

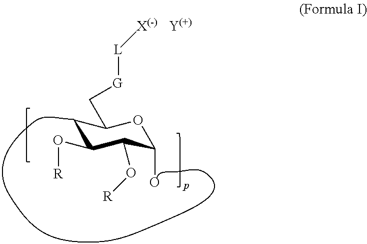

wherein

X⁽⁻⁾ is one or more negatively charged moieties,

Y⁽⁺⁾ is one or more counter cations,

L is one or more linkers,

G is a bond or is one or more bridging groups,

p is an integer, and

R is one or more substituents,

together with a pharmaceutically acceptable diluent.

The charged moiety X⁽⁻⁾ can be any suitable negatively charged moiety. Non-limiting examples include —SO₃⁻, —CO₂⁻, —OSO₃⁻, —OPO₃⁻, for example.

The linker L can comprise a substituted or unsubstituted alkyl group (such as a C₁-C₁₁ alkyl group, for example), and/or a substituted or unsubstituted polyethylene glycol (PEG) group, or a combination of one or more alkyl groups and one or more PEG groups. In an exemplary embodiment, the PEG group is of the formula —CHZ(CH₂OCHZ)ₘCH₂— where Z is H or CH₃ and m is 1 to 20, for example; however, any suitable PEG group, if present, may be contemplated. In certain embodiments, L can comprise any unsubstituted or substituted alkyl group; for example, the alkyl group may be substituted with a PEG group. However, any suitable substituent may be contemplated. In other embodiments, L can comprise an unsubstituted or substituted PEG group; for example, the PEG group may be substituted with one or more alkyl groups. However, any suitable substituent may be contemplated. In certain other embodiments, L comprises a PEG group which has none, or one or more alkyl groups flanking on either or both sides of the PEG group. One or more of the CH₂ groups of the alkyl group may be replaced with an atom or functional group. Non-limiting examples of the atom or functional group include —O—, —S—, —SO—, —SO₂—, —CONH—, —COO—, —NZ—, or a substituted or unsubstituted 1,2,3-triazole group, for example. Examples of substituted 1,2,3-triazole groups may include those substituted with a group comprising one of the following structures:

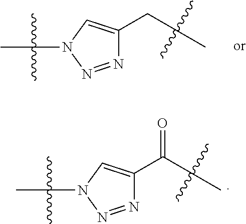 [SMILES: *Cc1cn(*)nn1 ; *C(=O)c1cn(*)nn1]

The cyclodextrin in the compound can comprise, for example, 6, 7, or 8 glucose subunits, typically 7. Thus, p can be 6, 7 or 8, typically 7.

In certain embodiments, G represents any one or more suitable bridging groups. G may represent, for example, an ester, amide, amine, sulfur, or a substituted or unsubstituted 1,2,3-triazole. Non-limiting examples of bridging groups for G include —S—, —OC(O)—, —NHC(O)—, —SO—, —SO₂—, or a substituted or unsubstituted 1,2,3-triazole group. Examples of substituted 1,2,3-triazole groups may include those substituted with a group comprising one of the following structures:

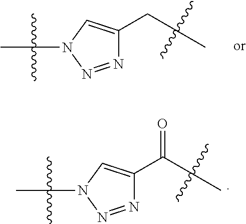 [SMILES: *Cc1cn(*)nn1 ; *C(=O)c1cn(*)nn1]

However, other suitable bridging groups may be contemplated. In certain other embodiments, G is a bond.

The substituent R can be any one or more suitable substituents. Non-limiting examples include H, an optionally substituted alkyl group or an optionally substituted acyl group. In certain embodiments, the optionally substituted alkyl group or acyl group is a C₁-C₁₈ group, for example.

Y⁽⁺⁾ can be any pharmaceutically acceptable cation, typically Na⁺ or K⁺, for example.

In accordance with another aspect of the present invention there is provided a pharmaceutical composition comprising a non-ionic cyclodextrin-based compound of the formula:

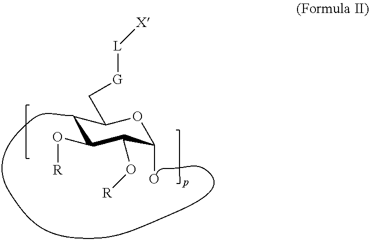

wherein

X′ is one or more neutral moieties,

L is one or more linkers,

G is a bond or is one or more bridging groups,

p is an integer, and

R is one or more substituents,

together with a pharmaceutically acceptable diluent.

Non-limiting examples of neutral moiety X′ may include, for example, an unsubstituted or substituted amide including its N-substituted forms (such as —CONH₂, for example), a nitrile group (—CN), or a polyhydroxylated residue (such as a carbohydrate for example).

The linker L can comprise a substituted or unsubstituted alkyl group (such as a C₁-C₁₁ alkyl group, for example), and/or a substituted or unsubstituted polyethylene glycol (PEG) group, or a combination of one or more alkyl groups and one or more PEG groups. In an exemplary embodiment, the PEG group is of the formula —CHZ(CH₂OCHZ)ₘCH₂— where Z is H or CH₃ and m is 1 to 20, for example; however, any suitable PEG group, if present, may be contemplated. In certain embodiments, L can comprise any unsubstituted or substituted alkyl group; for example, the alkyl group may be substituted with a PEG group. However, any suitable substituent may be contemplated. In other embodiments, L can comprise an unsubstituted or substituted PEG group; for example, the PEG group may be substituted with one or more alkyl groups. However, any suitable substituent may be contemplated. In certain other embodiments, L comprises a PEG group which has none, or one or more alkyl groups flanking on either or both sides of the PEG group. One or more of the CH₂ groups of the alkyl group may be replaced with an atom or functional group. Non-limiting examples of the atom or functional group include —O—, —S—, —SO—, —SO₂—, —CONH—, —COO—, —NZ—, or a substituted or unsubstituted 1,2,3-triazole group, for example. Examples of substituted 1,2,3-triazole groups may include those substituted with a group comprising one of the following structures:

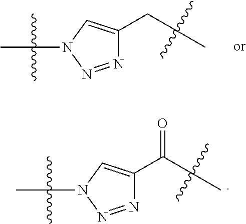 [SMILES: *Cc1cn(*)nn1 ; *C(=O)c1cn(*)nn1]

The cyclodextrin in the compound can comprise, for example, 6, 7, or 8 glucose subunits, typically 7. Thus, p can be 6, 7 or 8, typically 7.

In certain embodiments, G represents any one or more suitable bridging groups. G may represent, for example, an ester, amide, amine, sulfur, or a substituted or unsubstituted 1,2,3-triazole. Non-limiting examples of bridging groups for G include —S—, —OC(O)—, —NHC(O)—, —SO—, —SO₂—, or a substituted or unsubstituted 1,2,3-triazole group. Examples of substituted 1,2,3-triazole groups may include those substituted with a group comprising one of the following structures:

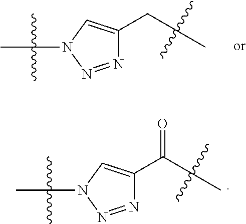 [SMILES: *Cc1cn(*)nn1 ; *C(=O)c1cn(*)nn1]

However, other suitable bridging groups may be contemplated. In certain other embodiments, G is a bond.

The substituent R can be any one or more suitable substituents. Non-limiting examples of R include H, an optionally substituted alkyl group or an optionally substituted acyl group. In certain embodiments, the optionally substituted alkyl group or acyl group is a C₁-C₁₈ group, for example.

In certain embodiments, the present application provides a polyanionic cyclodextrin-based compound as described herein, wherein p is 6 (α-cyclodextrin), 7 (β-cyclodextrin) or 8 (γ-cyclodextrin), X⁽⁻⁾ is —CO₂⁻ or —SO₃⁻; G is —S—; L is —(CH₂)ₖ—, where k is 1 to 11, optionally 7 to 11; or L is

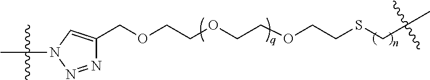

where q is 0 to 20 and n is 1-5, optionally 1-11, or

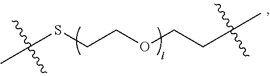

where 1 is 1-20; and R is H, optionally substituted C₁-C₁₈ alkyl, or optionally substituted C₁-C₁₈ acyl.

The compounds as described herein can be used in various pharmaceutical applications, such as excipients or by inclusion with guest molecules, such as for use as rescue medicines to remove undesired drugs and/or metabolites thereof.

The present application provides pharmaceutical compositions comprising a compound as substantially described herein together with a diluent. A compound as described herein can be used, for example, as an excipient or as a rescue medicine, such as for removing a compound from an organism, such as a human subject. The present application also provides a method of treating a subject in need thereof of an undesired molecule comprising administering a compound as described herein to said subject, such that the compound binds to said molecule, and removes it from said subject.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 shows an exemplary representation of thioether-linked polyanionic CDs with an additional PEG-ylated linker group.

FIG. 2 shows exemplary polyanionic sulfoPEG thioether CDs containing either two or three repeating units of PEG chains.

FIG. 3 shows exemplary water-soluble or amphiphilic polyanionic CDs containing PEG linkers.

FIG. 4 shows an exemplary polynon-ionic thioether CD analogs.

FIG. 5 shows exemplary polynon-ionic thioether CD polyamides.

FIG. 6 shows exemplary representation of water-soluble or amphiphilic polyanionic CDs containing thioether-linked sulfoalkyl groups.

FIG. 7 shows an inclusion study with polyanionic gamma-CD derivatives (structure 3) with rocuronium bromide by ¹H NMR spectroscopy.

FIG. 8 shows another inclusion study with polyanionic gamma-CD derivatives (structure 6) with rocuronium bromide by ¹H NMR spectroscopy.

FIG. 9 shows an inclusion study with polyanionic gamma-CD derivatives (structure 3) with Doxorubicin by ¹H NMR spectroscopy.

FIG. 10 shows an inclusion study with polyanionic gamma-CD derivatives (structure 6) with Tamoxifen citrate by ¹H NMR spectroscopy.

FIG. 11 shows an inclusion study with polynon-ionic gamma-CD derivatives (structure 14) with Tamoxifen citrate by ¹H NMR spectroscopy.

FIG. 12 shows an inclusion study with polyanionic gamma-CD derivatives (structure 6) with Diltiazem by ¹H NMR spectroscopy.

FIG. 13 shows another inclusion study with polynon-ionic gamma-CD derivatives (structure 11) with Diltiazem by ¹H NMR spectroscopy.

FIG. 14 shows another inclusion study with polynon-ionic gamma-CD derivatives (structure 14) with Diltiazem by ¹H NMR spectroscopy.

FIG. 15 shows an inclusion study with polyanionic gamma-CD derivatives (structure 3) with Naloxine hydrochloride by ¹H NMR spectroscopy.

FIG. 16 shows an inclusion study with polyanionic gamma-CD derivatives (structure 3) with Valsartan by ¹H NMR spectroscopy.

FIG. 17 shows an inclusion study with polyanionic beta-CD derivatives (structure 2) with Carprofen by ¹H NMR spectroscopy.

FIG. 18 shows an inclusion study with polyanionic beta-CD derivatives (structure 2) with Flurbiprofen by ¹H NMR spectroscopy.

FIG. 19 shows an inclusion study with polyanionic gamma-CD derivatives (structure 3) with Naftifine HCl by ¹H NMR spectroscopy.

FIG. 20 shows an inclusion study with polyanionic gamma-CD derivatives (structure 3) with Oxytetracycline HCl by ¹H NMR spectroscopy.

FIG. 21 shows an inclusion study with polyanionic gamma-CD derivatives (structure 3) with Doxycycline Hyclate by ¹H NMR spectroscopy.

FIG. 22 shows an inclusion study with polyanionic gamma-CD derivatives (structure 3) with Amitriptyline HCl by ¹H NMR spectroscopy.

FIG. 23 shows an inclusion study with polyanionic gamma-CD derivatives (structure 3) with Acebutolol HCl by ¹H NMR spectroscopy.

FIG. 24 shows an inclusion study with polyanionic beta-CD derivatives (structure 2) with Bupivacaine, HCl by ¹H NMR spectroscopy.

FIG. 25 shows an inclusion study with polyanionic beta-CD derivatives (structure 2) with Ipratropium Bromide by ¹H NMR spectroscopy.

FIG. 26 shows an inclusion study with polyanionic beta-CD derivatives (structure 2) with Tiquizium bromide by ¹H NMR spectroscopy.

FIG. 27 illustrates NMR results for the inclusion of Nefopam hydrochloric acid with structure 3.

FIG. 28 illustrates NMR results for the inclusion of Clomipramine hydrochloric acid with structure 3.

FIG. 29 illustrates NMR results for the inclusion of Isoconazole nitrate with structure 3.

FIG. 30 illustrates NMR results for the inclusion of Voriconazole with structure 3.

FIG. 31 illustrates NMR results for the inclusion of Butoconazole nitrate with structure 3.

FIG. 32 illustrates NMR results for the inclusion of Imazalil sulfate with structure 3.

FIG. 33 illustrates NMR results for the inclusion of Ziprasidone hydrochloric acid with structure 3.

FIG. 34 illustrates NMR results for the inclusion of Econazole with structure 3.

FIG. 35 illustrates NMR results for the inclusion of sertaconazole nitrate with structure 3.

FIG. 36 illustrates NMR results for the inclusion of irinotecan HCl with structure 3.

FIG. 37 shows structures of selected commercial drugs used for inclusion studies with polyanionic gamma-CD derivatives (structure 3 and 6) by Electrospray Ionization Mass Spectrometry.

FIG. 38 shows an inclusion study with polyanionic gamma-CD derivatives (structure 3) with Rocuronium Bromide by Electrospray Ionization Mass Spectrometry.

FIG. 39 shows an inclusion study with polyanionic gamma-CD derivatives (structure 6) with Rocuronium Bromide by Electrospray Ionization Mass Spectrometry.

FIG. 40 shows Kd,app for CDs structure 3 (PZ7095) and structure 6 (PZ7086) binding to various drugs measured by ESI-MS in 10 mM ammonium acetate, pH 6.8.

FIG. 41 shows Kd,app for CD (structure 3, PZ7095) binding to various drugs measured by ESI-MS in 10 mM ammonium acetate, pH 6.8.

FIG. 42 shows hemolysis results for polysulfonate structures 2, 3, 5 and 6.

FIG. 43 shows two examples of caroboxyPEG thioether CDs (structures 17 and 18) in accordance with the present invention.

FIG. 44 shows an exemplary synthesis of carboxyPEG thioether CD analogs (structures 17 and 18).

FIG. 45 illustrates NMR results for the inclusion of diltiazem with structure 18.

FIG. 46 illustrates NMR results for the inclusion of amitripline with structure 18.

FIG. 47 illustrates NMR results for the inclusion of clomipramine with structure 18.

FIG. 48 illustrates NMR results for the inclusion of tamoxifen citrate with structure 18.

FIG. 49 illustrates NMR results for the inclusion of toremifene citrate with structure 18.

FIG. 50 illustrates NMR results for the inclusion of voriconazole with structure 18.

### DETAILED DESCRIPTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

As used herein, the term “aliphatic” refers to a linear, branched or cyclic, saturated or unsaturated non-aromatic hydrocarbon. Examples of aliphatic hydrocarbons include alkyl groups.

As used herein, the term “alkyl” refers to a linear, branched or cyclic, saturated or unsaturated hydrocarbon group which can be unsubstituted or is optionally substituted with one or more substituent. Examples of saturated straight or branched chain alkyl groups include, but are not limited to, methyl, ethyl, 1-propyl, 2-propyl, 1-butyl, 2-butyl, 2-methyl-1-propyl, 2 methyl 2-propyl, 1 pentyl, 2-pentyl, 3-pentyl, 2-methyl-1-butyl, 3-methyl-1-butyl, 2 methyl-3-butyl, 2,2 dimethyl 1-propyl, 1-hexyl, 2-hexyl, 3-hexyl, 2-methyl-1-pentyl, 3 methyl-1-pentyl, 4 methyl-1-pentyl, 2-methyl-2-pentyl, 3-methyl-2-pentyl, 4 methyl 2 pentyl, 2,2 dimethyl 1 butyl, 3,3-dimethyl-1-butyl and 2-ethyl-1-butyl, 1-heptyl and 1-octyl. As used herein the term “alkyl” encompasses cyclic alkyls, or cycloalkyl groups. The term “cycloalkyl” as used herein refers to a non-aromatic, saturated monocyclic, bicyclic or tricyclic hydrocarbon ring system containing at least 3 carbon atoms. Examples of C3-C12 cycloalkyl groups include, but are not limited to, cyclopropyl, cyclobutyl, cyclopentyl, cyclohexyl, cycloheptyl, cyclooctyl, norbornyl, adamantyl, bicyclo[2.2.2]oct-2-enyl, and bicyclo[2.2.2]octyl. Chemical functional groups, such as ether, thioether, sulfoxide, or amine, amide, ammonium, ester, phenyl, 1,2,3-triazole etc can be incorporated alkyl group to help extend the length of the chain.

As used herein, the term “substituted” refers to the structure having one or more substituents. A substituent is an atom or group of bonded atoms that can be considered to have replaced one or more hydrogen atoms attached to a parent molecular entity. Examples of substituents include aliphatic groups, halogen, arylcarbonyloxy, alkoxycarbonyloxy, aryloxycarbonyloxy, alkoxycarbonyl, aminocarbonyl, alkylthiocarbonyl, alkoxyl, phosphate ester, phosphonato, phosphinato, cyano, tertiary amino, tertiary acylamino, tertiary amide, imino, alkylthio, arylthio, sulfonato, sulfamoyl, tertiary sulfonamido, nitrile, trifluoromethyl, heterocyclyl, aromatic, and heteroaromatic moieties, ether, ester, boron-containing moieties, tertiary phosphines, and silicon-containing moieties.

As used herein, the term “hydrophilic” refers to the physical property of a molecule or chemical entity or substituent within a molecule that tends to be miscible with and/or dissolved by water, or selectively interacts with water molecules. Hydrophilic groups can include polar groups. By contrast, as used herein, the term “hydrophobic” refers to the physical property of a molecule or chemical entity or substituent within a molecule that tends to be immiscible with and/or insoluble in water, or selectively repels water molecules.

As used herein, the term “amphiphilic” refers to the physical property of a molecule or chemical entity that possesses both hydrophilic and hydrophobic properties.

As used herein, the term “anionic” refers to a negatively charged molecule or part thereof which imparts the negative charge.

As used herein, an “excipient” refers to an inactive substance that serves as the vehicle or medium for a drug or other active substance in a pharmaceutical composition.

As used herein, a “rescue medicine” can refer to any compound or composition comprising said compound, which can be used to bind to another compound. Typically, the rescue medicine is for binding to and removing the other compound from an organism, such as a human subject. The other compound can be a drug or a metabolite thereof. In certain embodiments, the drug or metabolite thereof is undesired in the organism, is toxic, and/or is in excessive quantities in the organism.

In the present document, the hydrophobic groups are illustrated to be placed at the secondary face of a CD while the hydrophilic groups are placed at the primary face of a CD. These two groups can be swapped to link to the opposite face of a CD.

The present application provides the use of polyanionic and non-ionic CD-based compounds, ideally in a pure form, as carrier molecules for various guest molecules.

The present application provides a composition comprising a polyanionic or non-ionic CD-based compound for use as a rescue medicine. The compounds as described herein can be used as an excipient to associate with a number of guest molecules. The compounds can also be used, for example, in removing undesired drugs and/or metabolites thereof.

Ideally, the polyanionic and non-ionic CD-based compounds as described herein can use thioether or its oxidized form (sulfone or sulfoxide) as the linking group instead of ether as done previously in the art. This results in structurally well-defined polyanionic and non-ionic CD-based compounds in pure form that are easier to characterize. As such, the polyanionic and non-ionic CD-based compounds of the present application are suitable for generating drug formulations in well-defined compositions.

Advantageously, the present polyanionic and non-ionic CD-based compounds can bind to other molecules with better affinity due to the symmetric nature of the cavity within the CD. The cavity can accommodate larger or smaller molecules as the polyanionic or non-ionic CD can be an α, β, or γ analog.

The polyanionic and non-ionic CD-based compounds can be designed to be either totally water-soluble (with short chains, where R is H, methyl to n-propyl, or acetyl to n-propanoyl) or self-assemble (with longer chains, where R is n-butyl to n-octadecyl or n-butanoyl to n-octadecanoyl) to form nanoparticles (micelles) in water. These structures ideally bind to hydrophobic drug molecules with better affinities because of the alkyl chains and the PEG linker groups.

The number of linkers attached to the cyclodextrin can vary but are typically the same length within a given CD-based molecule.

The CD core (i.e., D) comprises any number of glucose subunits. In certain embodiments, there are 6, 7, or 8 glucose subunits, typically 7. Therefore, in certain embodiments, a β-CD is contemplated.

On the secondary face of the CD are attached one or more, typically a plurality of substituents, R. The substituents can be H, an alkyl or acyl group. In certain embodiments, the chains are bonded to either O2 or O3 of the CD group, or both O2 and O3 groups. The length of the group can vary from C₁-C₁₈, for example.

To gain a better understanding of the invention described herein, the following examples are set forth. It should be understood that these examples are for illustrative purposes only. Therefore, they should not limit the scope of this invention in any way.

### EXAMPLES

### Example 1: Water Soluble Polyanionic CD-Based Compounds

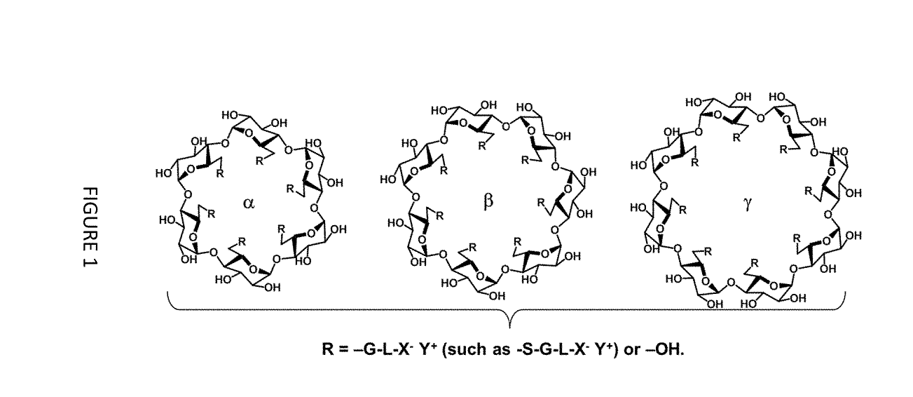

FIG. 1 shows α, β and γ embodiments of CDs as described herein. In these embodiments, the 6-hydroxyl groups of native cyclodextrins are partially or completely replaced with R groups of the formula -G-L-X⁻ Y⁺, -S-G-L-X⁻ Y⁺ or —OH. G, L, X and Y are as defined above.

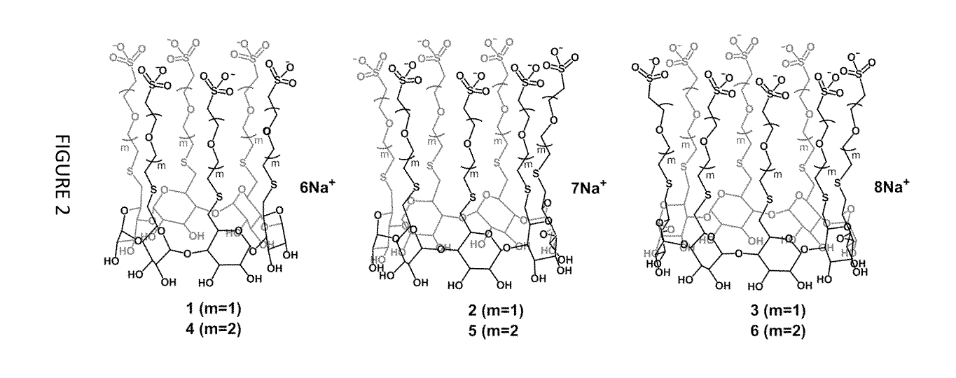

FIG. 2 shows examples of synthesized sulfoPEG thioether CD analogs (1-6). Left panel shows two α-CD derivatives (structures 1 and 4) containing different length of linker, middle panel shows two -CD analogs (2 and 5) and right panel show two γ-CD analogs (3 and 6). In each pair of example shown, the number of PEG group varies between two and three units; however, it may be contemplated as stated above that any number of PEG groups may be present.

### Example 2: Water-Soluble and Amphiphilic Polyanionic CD-Based Compounds

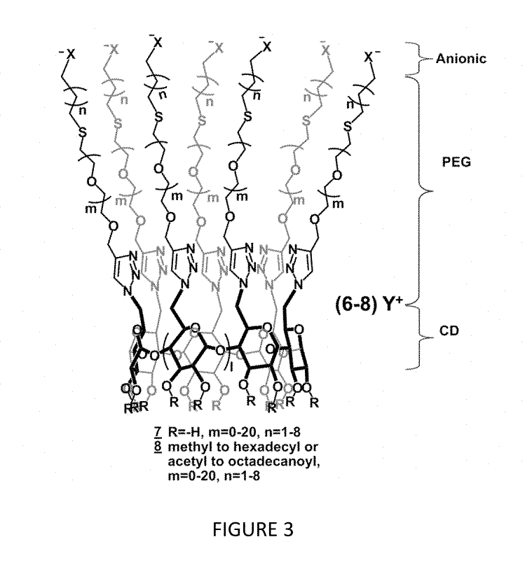

FIG. 3 shows an exemplary polyanionic CD with a PEG-ylated linker group. As shown, the anionic group can be any suitable group, such as —SO3- or —CO2- for example. The PEG segment can include 1 to 20 repeating ethylene glycol groups. Typically, the bridging group used to connect PEG segment to D-glucose is a substituted 1,2,3-triazole group such as the (1,2,3-triazole-4-yl)methyl or (1,2,3-triazole-4-yl)carbonyl group. The compound can be either a water-soluble polyionic cyclodextrin (R═H (structure 7), methyl to n-butyl (structure 8)) or capable of self-assembling in water (R=longer than n-butyl, structure 8). Y⁺ can be N⁺, K⁺ or any other pharmaceutically tolerated cation.

### Example 3: Non-Ionic CD-Based Compounds

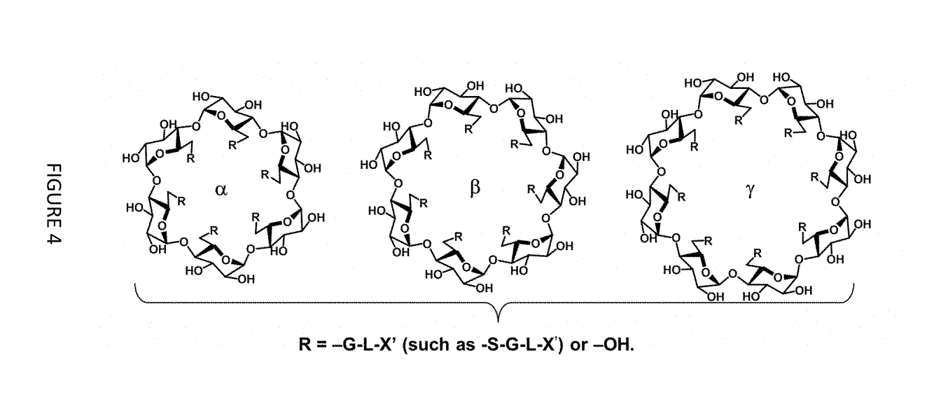

FIG. 4 shows α, β and γ embodiments of CDs as described herein. In these embodiments, the 6-hydroxyl groups of native cyclodextrins are partially or completely replaced with R groups of the formula -G-L-X′, such as -S-L-X′, or with —OH. G, L and X′ are defined above.

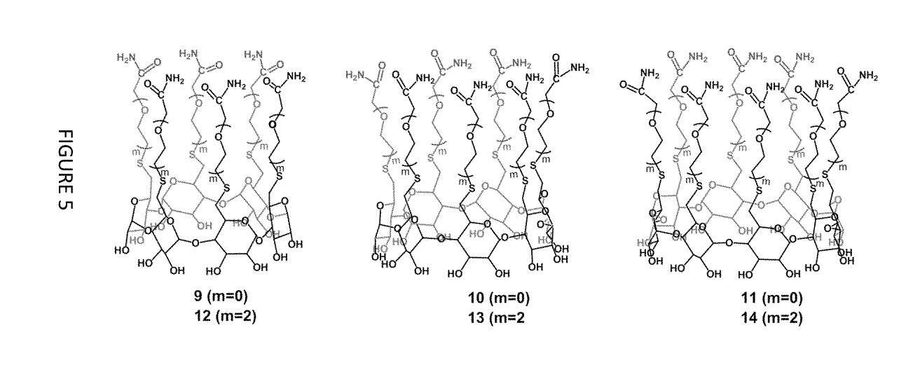

FIG. 5 shows examples of synthesized non-ionic CD-based thioether polyamides (9-14). Left panel shows two α-CD derivatives (structures 9 and 12) containing different length of linker. Middle panel shows two f-CD analogs (10 and 13) and right panel show two γ-CD analogs (11 and 14). In each pair of example shown, the embedded number of PEG group was either none or two units; however, it may be contemplated as stated above that any number of PEG groups may be present.

### Example 4: Thioether-Linked Sulfoalkyl Polyanionic CDs

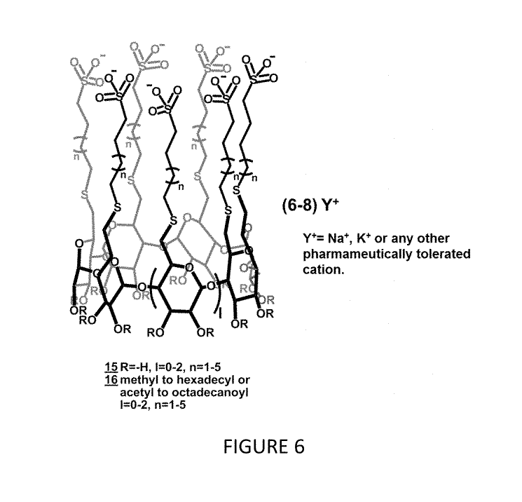

FIG. 6 shows an exemplary thioether-linked sulfoalkyl polyanionic CD-based compound. The molecule comprises a saturation of the CD groups with an alkyl linker typically, propyl (trimethylene) or butyl (tetramethylene) and thioether as the bridging functionality to connect the linkers to CD. The length of the linker can vary. Exemplary R groups on the secondary face of the CD are shown.

### Example 4: Inclusion Experiments with Commercial Medicines Using NMR

Inclusion studies were conducted to determine whether the CD-based polyanionic SulfoPEG thioether and non-ionic thioether polyamides described herein are suitable for carrying out inclusion with different families of drug molecules.

Rocuronium bromide, Pipecuronium bromide, Pancuronium bromide and Vecuronium bromide belong to a family of aminosteroids that act as non-depolarizing neuromuscular blockers. They are used in modern anaesthesia. Molecular hosts capable of complexing aminosteroids may reverse the effects of administered aminosteroid.

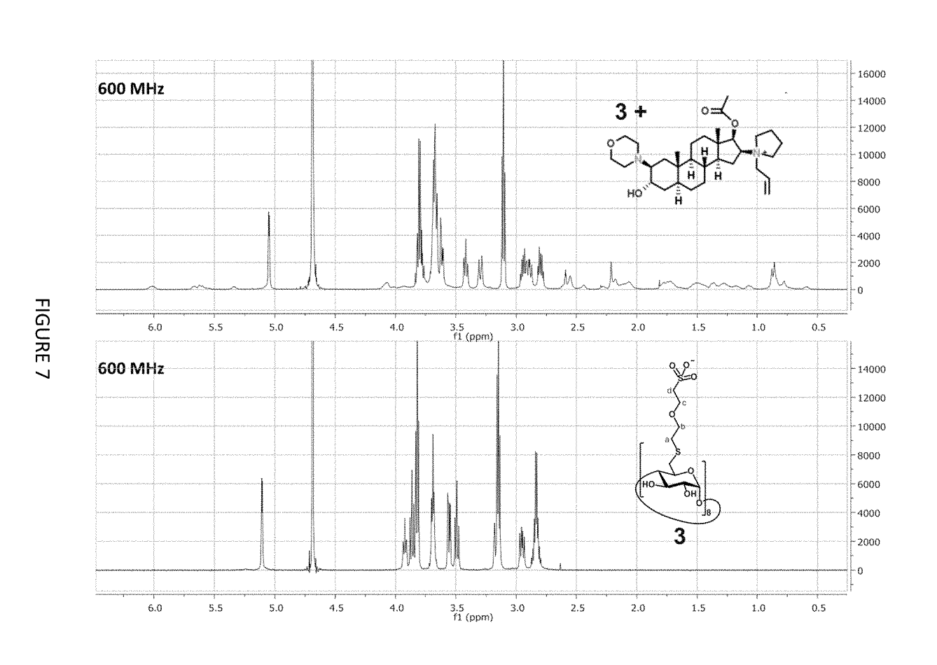

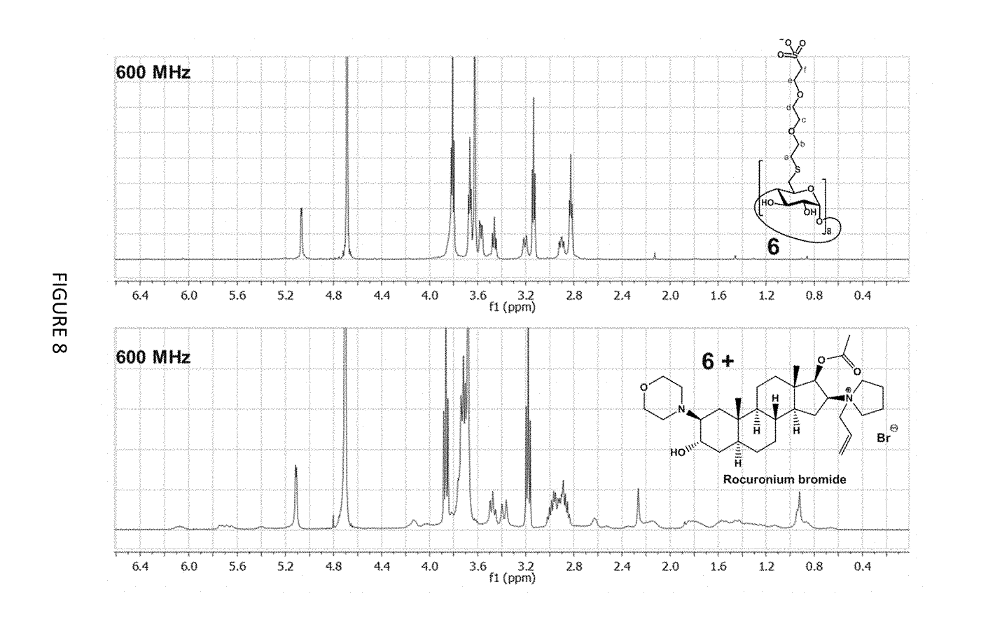

FIG. 7 shows an inclusion study of sulfoPEG gamma-CD derivative 3 with rocuronium bromide by NMR experiments (bottom panel: compound 3 alone, top panel: compound 3 with rocuronium bromide). FIG. 8 shows additional inclusion studies of compound 6 with rocuronium bromide (top panel: compound 6 alone, bottom panel: compound 6 with Rocuronium bromide). Significant changes in chemical shifts were observed for both CD molecules, suggesting interaction of guest molecule with CD cavity and PEG chains. Thus, polyanionic CD compounds in accordance with the present invention can be used to form an inclusion complex with Rocuronium bromide, and might be applicable for use with analogs thereof.

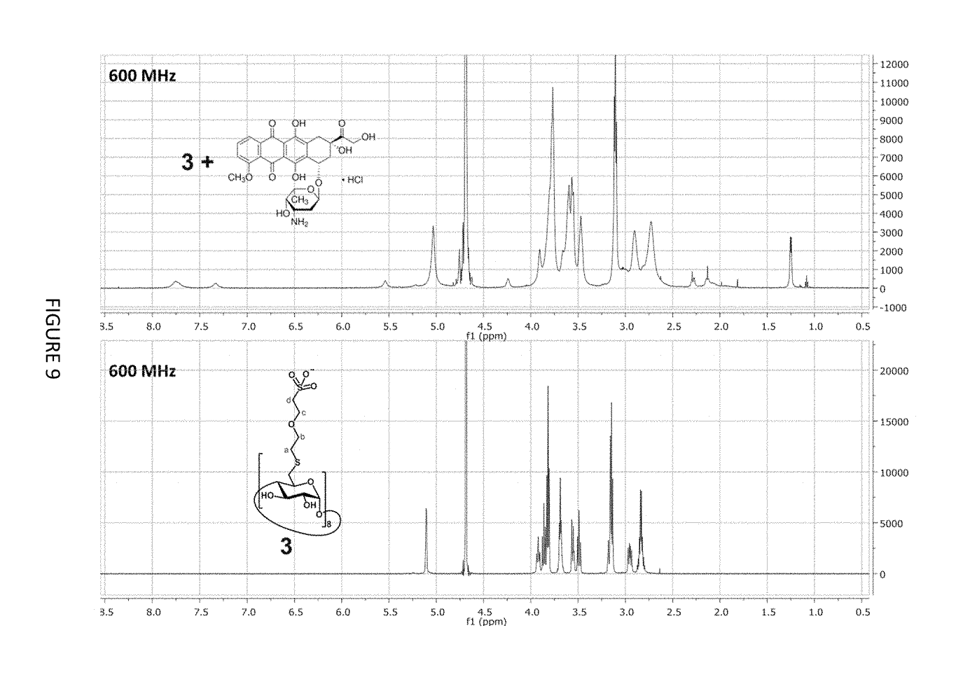

Doxorubicin hydrochloride is an anti-cancer chemotherapy drug. FIG. 9 shows inclusion studies between the polyanionic gamma-CD 3 and Doxorubicin hydrochloride by NMR experiment (bottom panel: compound 3 alone, top panel: compound 3 with Doxorubicin hydrochloride). Significant changes in chemical shifts of host CD molecule (3) before and after mixing with Doxorubicin were found, suggesting the CD host 3 can form an inclusion complex with doxorubicin.

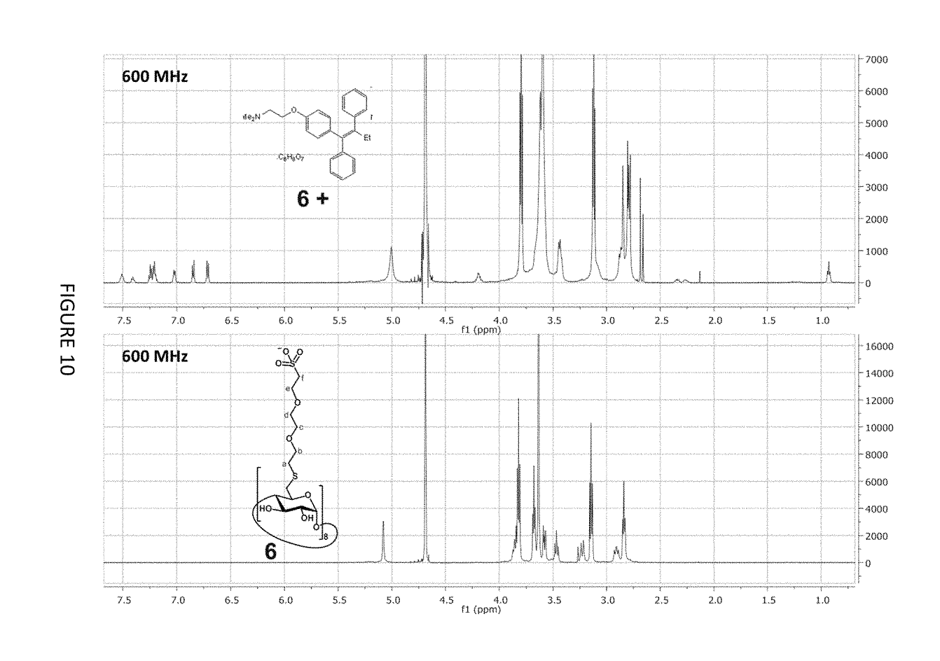

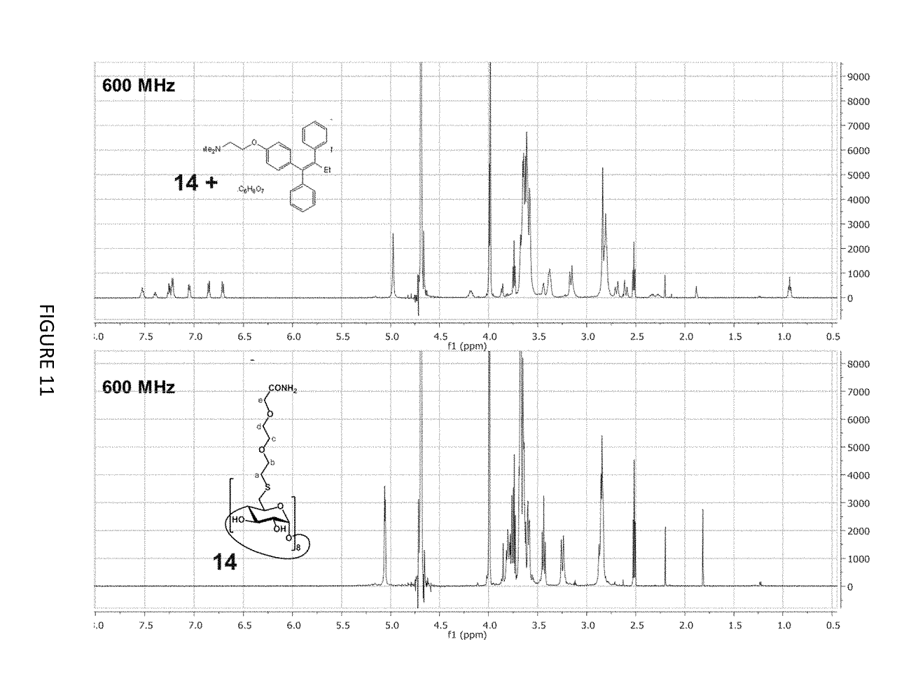

Tomoxifen citrate is another anti-cancer chemotherapy drug. FIG. 10 shows inclusion studies between the polyanionic gamma-CD 3 and Tomoxifen citrate by NMR (bottom panel: compound 3 alone, top panel: compound 3 with Tomoxifen citrate). FIG. 11 shows an additional inclusion study between non-anionic thioether gamma-CD polyamide 14 and Tomoxifen citrate. Significant changes in chemical shifts of host CD molecules (3 and 14) before and after mixing with Tomoxifen citrate were observed, suggesting both the polyanionic and non-ionic CD hosts in accordance with the present invention can be used to form an inclusion complex with Tomoxifen and might be applicable for use with analogs thereof.

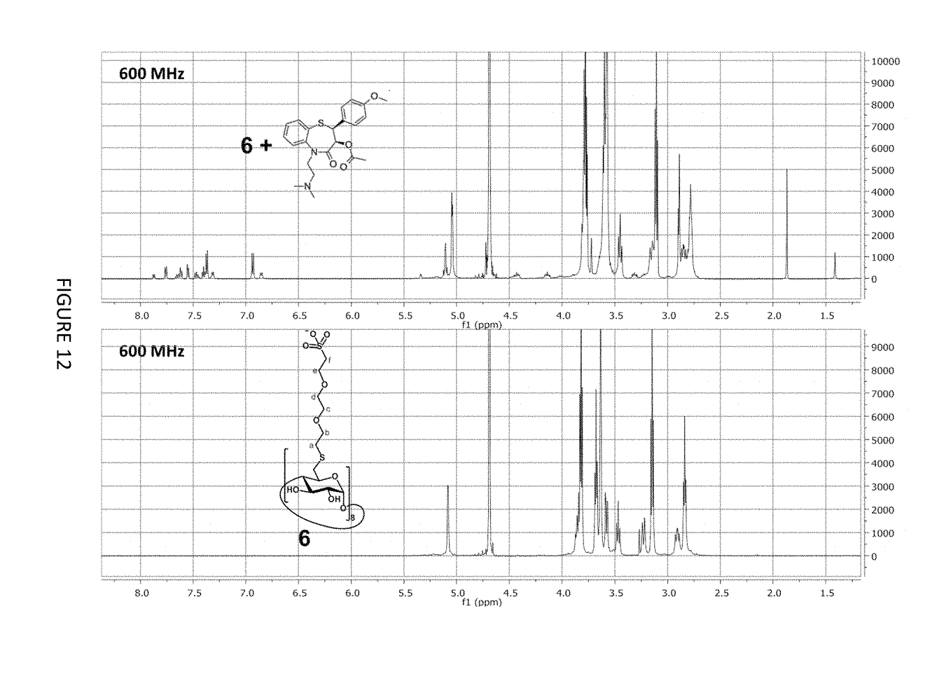

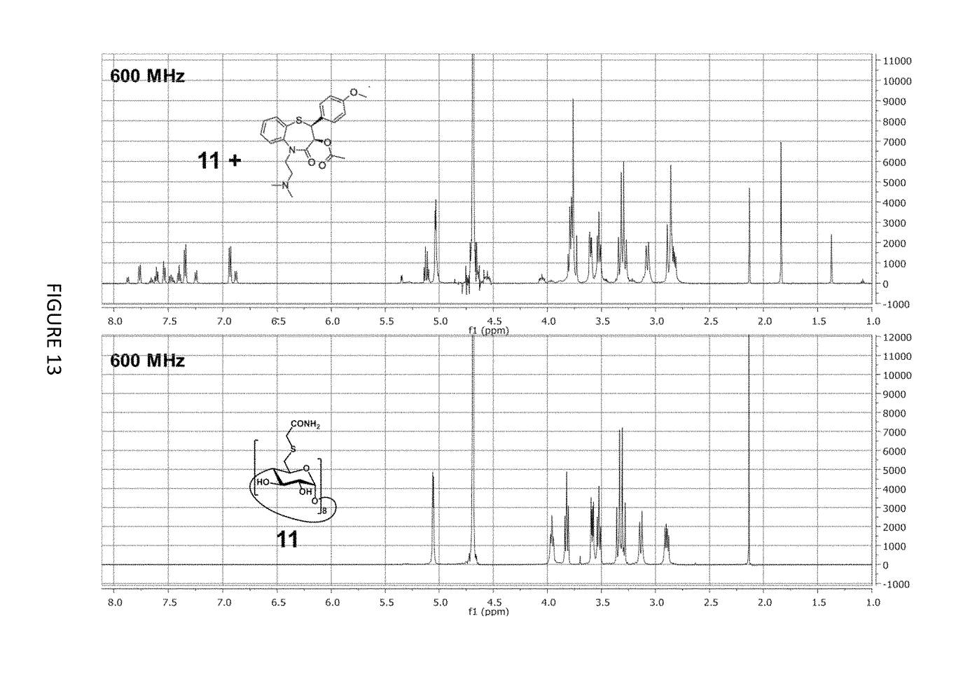

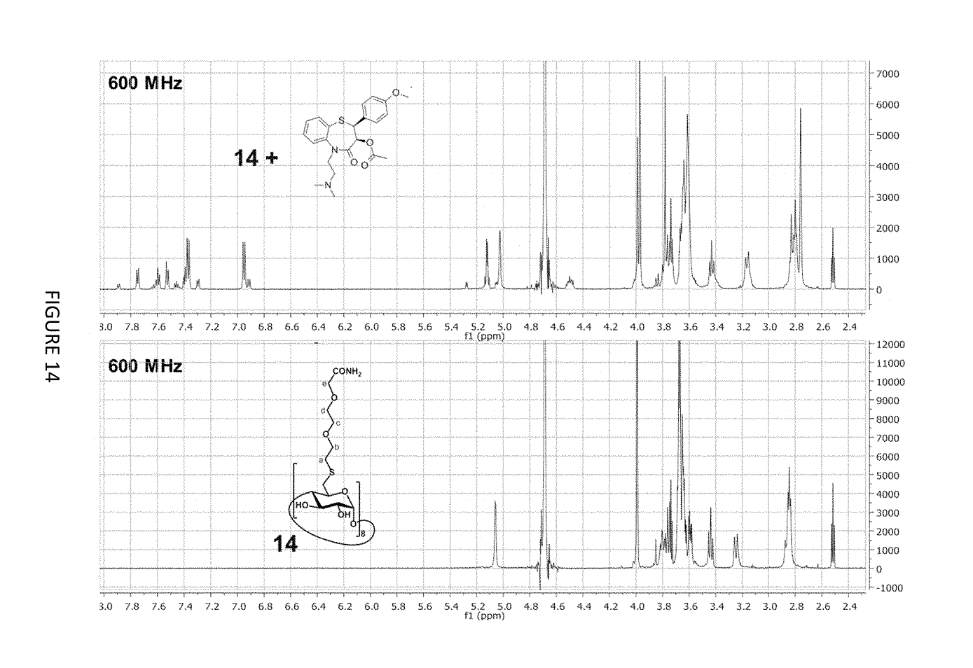

Diltiazem hydrochloride is in a class of medications called calcium-channel blockers and it is used to treat high blood pressure and to control angina (chest pain). FIG. 12 shows inclusion studies between the polyanionic gamma-CD 6 and Diltiazem hydrochloride by NMR (bottom panel: compound 6 alone, top panel: compound 6 with Diltiazem hydrochloride). FIG. 13 shows additional inclusion studies between non-anionic thioether gamma-CD polyamide 11 with Diltiazem hydrochloride by NMR experiment (bottom panel: compound 11 alone, top panel: compound 11 with Diltiazem hydrochloride). FIG. 14 shows an additional inclusion studies between non-anionic thioether gamma-CD polyamide 14 with Diltiazem hydrochloride by NMR (bottom panel: compound 14 alone, top panel: compound 14 with Diltiazem hydrochloride). In all cases, significant changes in chemical shifts of host CD molecules (3, 11 and 14) before and after mixing with Diltiazem hydrochloride were observed, suggesting both the polyanionic and non-ionic CD hosts in accordance with the present invention can be used to form inclusion complexes with Diltiazem and might be applicable for use with analogs thereof.

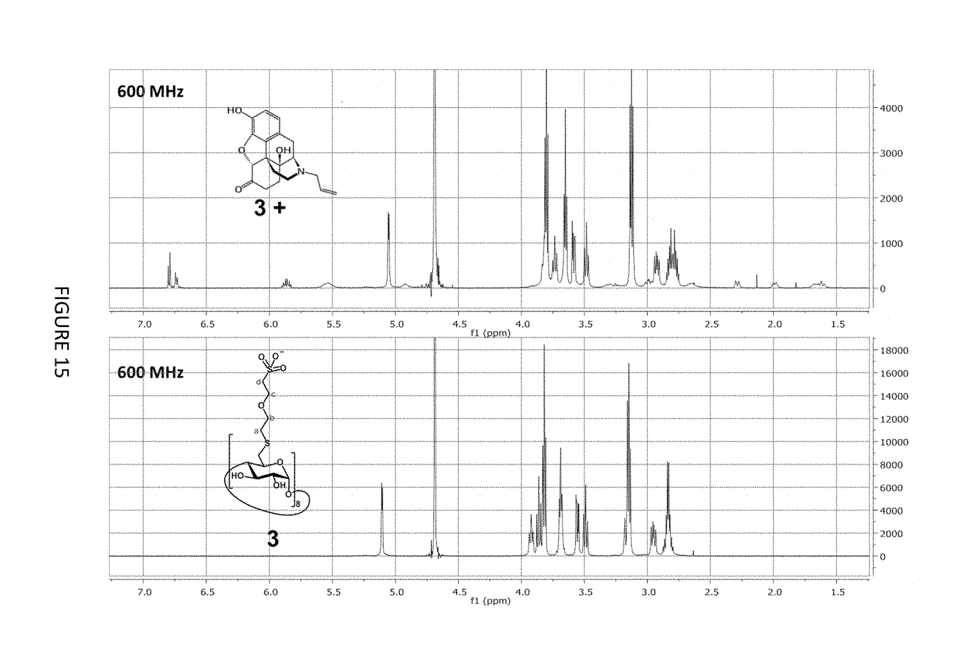

Naloxone is used to reverse the effects of narcotic drugs used during surgery or to treat pain. FIG. 15 shows inclusion studies between the polyanionic gamma-CD 3 and Naloxone hydrochloride by NMR (bottom panel: compound 3 alone, top panel: compound 3 with Naloxone hydrochloride). Significant changes in chemical shifts of host CD molecule (3) before and after mixing with Naloxone hydrochloride were observed, suggesting the polyanionic CD hosts in accordance with the present invention can be used to form inclusion complexes with Naloxone and might be applicable for use with related narcotics.

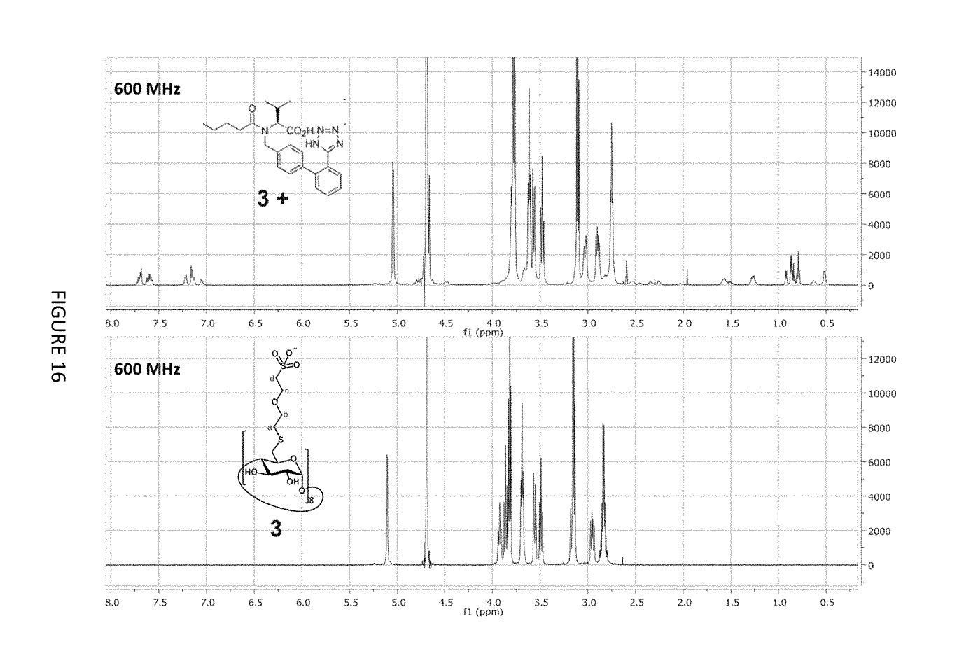

Valsartan is used to treat high blood pressure and congestive heart failure. FIG. 16 shows inclusion studies between the polyanionic gamma-CD 3 and Valsartan by NMR (bottom panel: compound 3 alone, top panel: compound 3 with Valsartan). Significant changes in chemical shifts of host CD molecule (3) before and after mixing with Valsartan were observed, suggesting the polyanionic CD hosts in accordance with the present invention can be used to form inclusion complexes with Valsartan and might be applicable for use with analogs thereof.

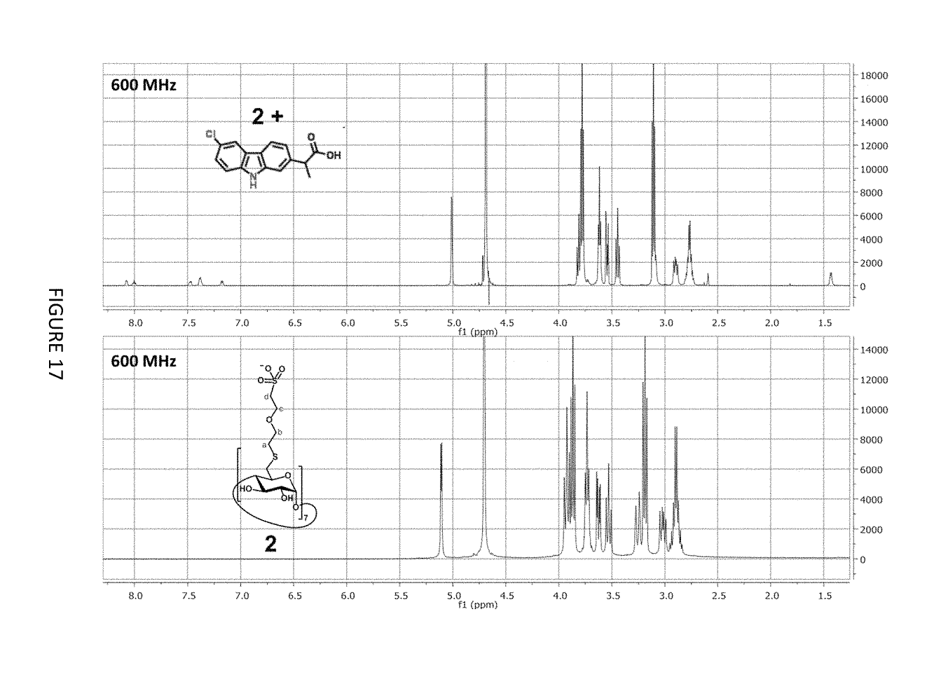

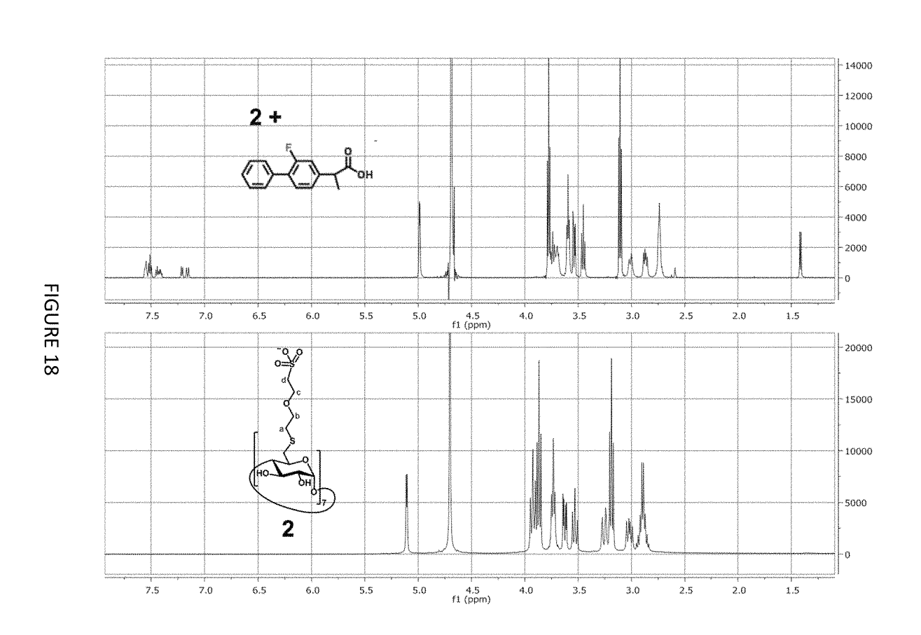

Carprofen is a non-narcotic, non-steroidal anti-inflammatory agent with characteristic analgesic and antipyretic activity. Flurbiprofen is another drug of the same family prescribed to treat inflammation and pain of certain arthritic conditions and soft tissue injuries. FIG. 17 shows inclusion studies between the polyanionic beta-CD 2 and Carprofen by NMR (bottom panel: compound 2 alone, top panel: compound 2 with Carprofen), and FIG. 18 shows inclusion studies between the polyanionic beta-CD 2 and Flurbiprofen by NMR (bottom panel: compound 2 alone, top panel: compound 2 with Flurbiprofen). Significant changes in chemical shifts of host CD molecule (2) before and after mixing with either Carprofen or Flurbiprofen were observed, suggesting the polyanionic CD hosts in accordance with the present invention can be used to form inclusion complexes with Carprofen or Flurbiprofen, and might be applicable for use with related analogs thereof.

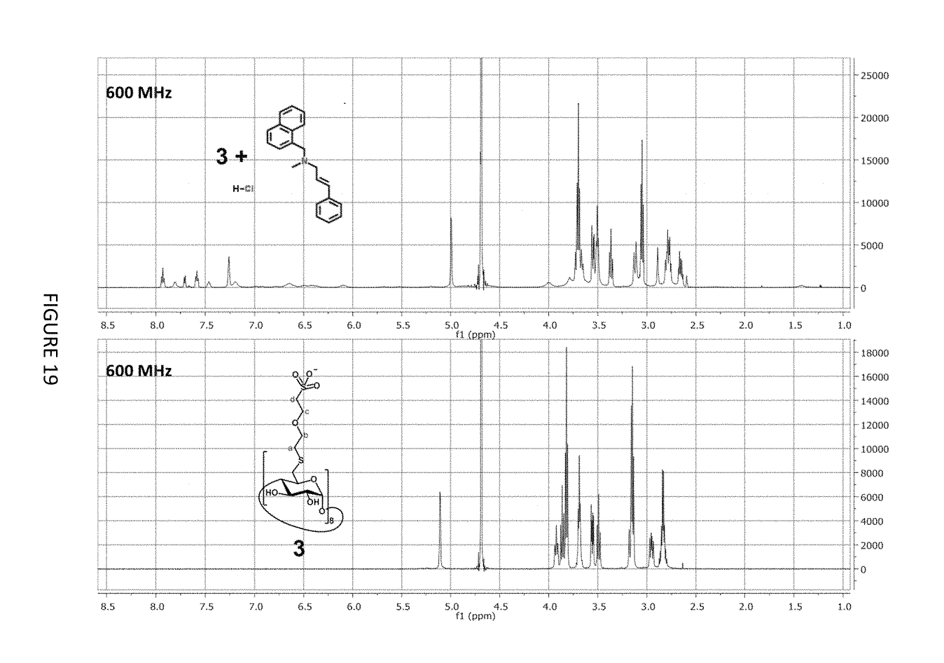

Naftifine hydrochloride is an antifungal medicine used in the treatment of skin infections. FIG. 19 shows inclusion studies between the polyanionic gamma-CD 3 and Naftifine hydrochloride by NMR (bottom panel: compound 3 alone, top panel: compound 3 with Naftifine hydrochloride). Significant changes in chemical shifts of host CD molecule (3) before and after mixing with Naftifine hydrochloride were observed, suggesting the polyanionic CD hosts in accordance with the present invention can be used to form inclusion complexes with Naftifine hydrochloride and might be applicable for use with related analogs thereof.

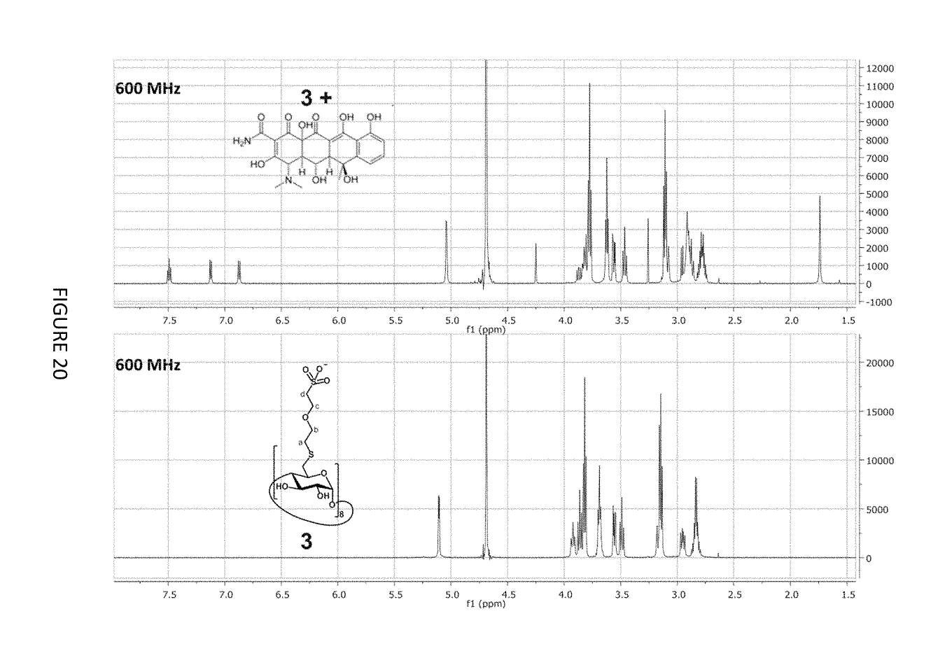

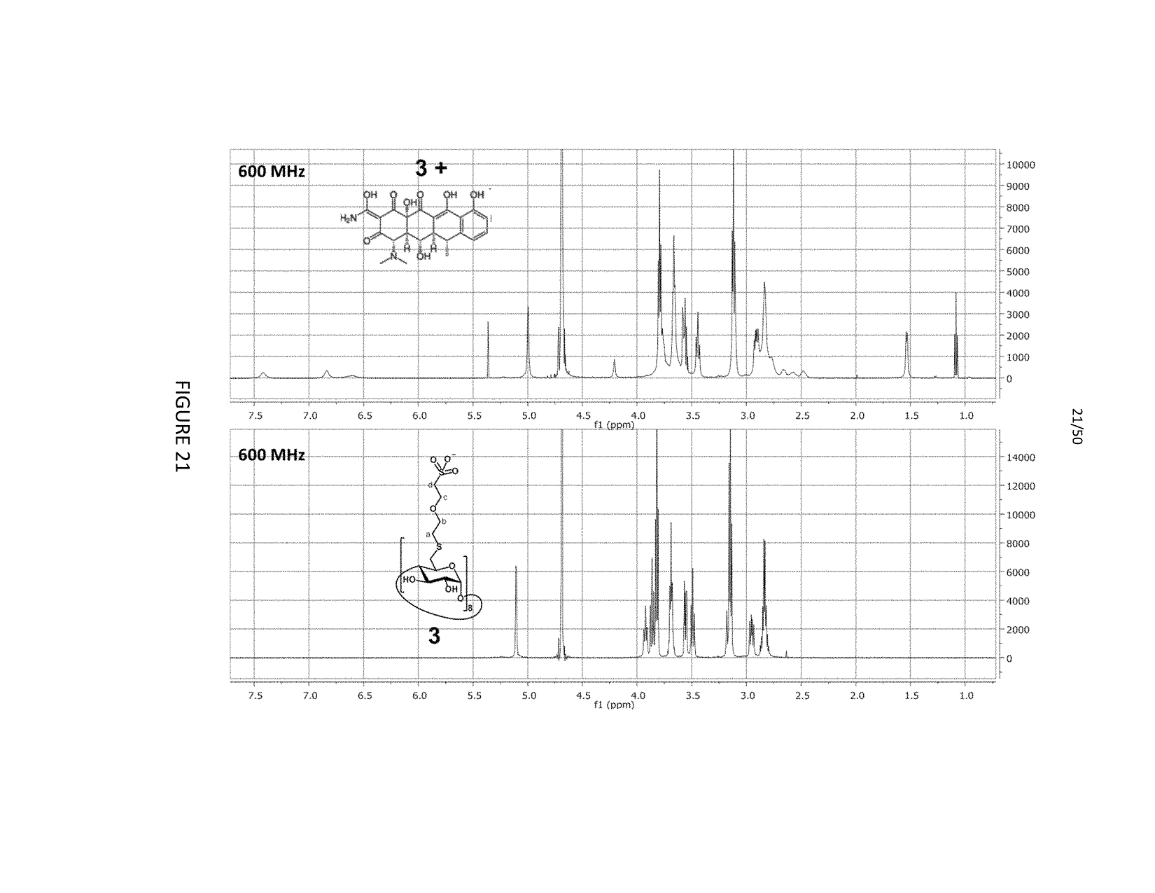

Oxytetracycline hydrochloride and Doxycycline Hyclate are both antibacterial agents of the tetracycline families. FIG. 20 shows inclusion studies between the polyanionic gamma-CD 3 and Oxytetracycline hydrochloride by NMR (bottom panel: compound 3 alone, top panel: compound 3 with Oxytetracycline hydrochloride), and FIG. 21 shows inclusion studies between the polyanionic gamma-CD 3 and Doxycycline Hyclate by NMR (bottom panel: compound 3 alone, top panel: compound 3 with Doxycycline Hyclate). In both cases, significant changes in chemical shifts of host CD molecule (3) before and after mixing with the tetracycline derivative were observed, suggesting the polyanionic CD hosts in accordance with the present invention can be used to form inclusion complexes with Oxytetracycline hydrochloride and Doxycycline Hyclate and might be applicable for use with related analogs thereof.

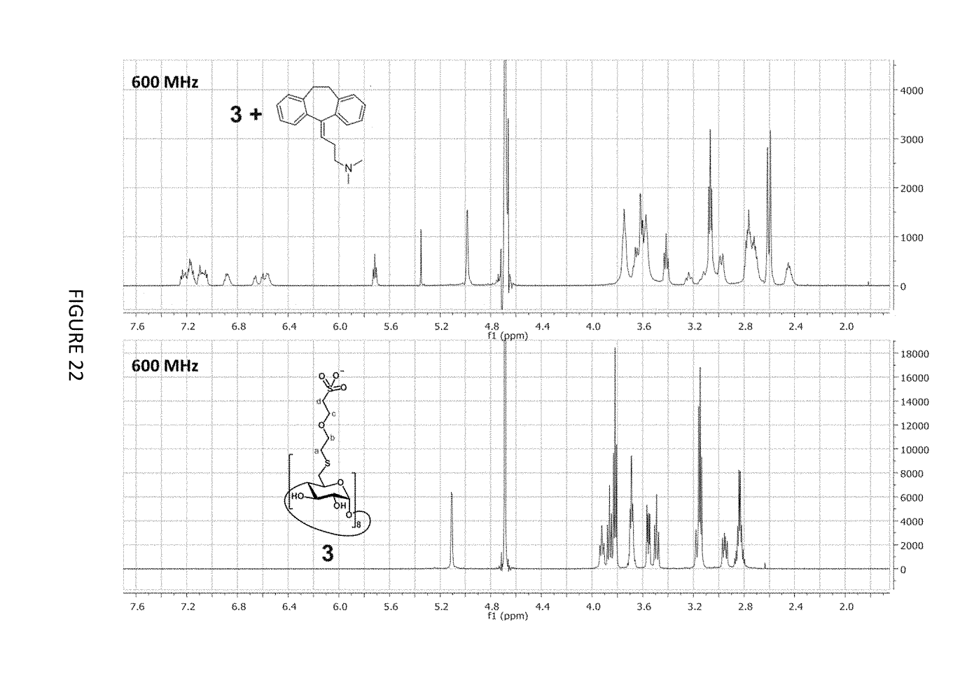

Amitriptyline hydrochloride is a tricyclic antidepressant and is used to treat symptoms of depression. FIG. 22 shows inclusion studies between the polyanionic gamma-CD 3 and Amitriptyline hydrochloride by NMR (bottom panel: compound 3 alone, top panel: compound 3 with Amitriptyline hydrochloride). Significant changes in chemical shifts of host CD molecule (3) before and after mixing with Amitriptyline hydrochloride were observed, suggesting the polyanionic CD hosts in accordance with the present invention can be used to form inclusion complexes with Amitriptyline hydrochloride and might be applicable for use with related analogs thereof.

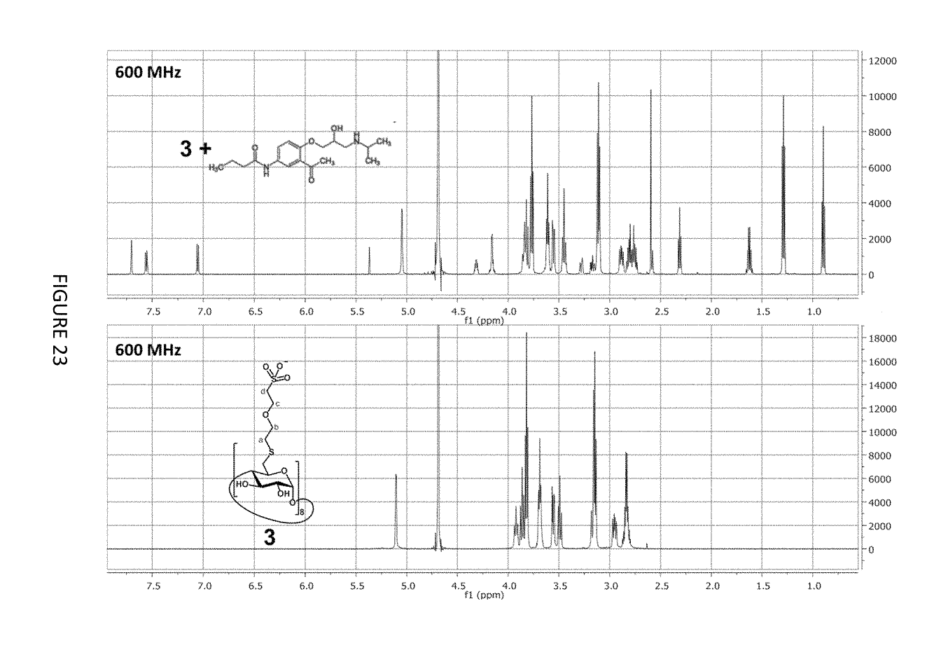

Acebutolol hydrochloride a used to treat patients with hypertension and ventricular arrhythmias. FIG. 23 shows inclusion studies between the polyanionic gamma-CD 3 and Acebutolol hydrochloride by NMR (bottom panel: compound 3 alone, top panel: compound 3 with Acebutolol hydrochloride). Significant changes in chemical shifts of host CD molecule (3) before and after mixing with Acebutolol hydrochloride were observed, suggesting the polyanionic CD hosts in accordance with the present invention can be used to form inclusion complexes with Acebutolol hydrochloride and might be applicable for use with related analogs thereof.

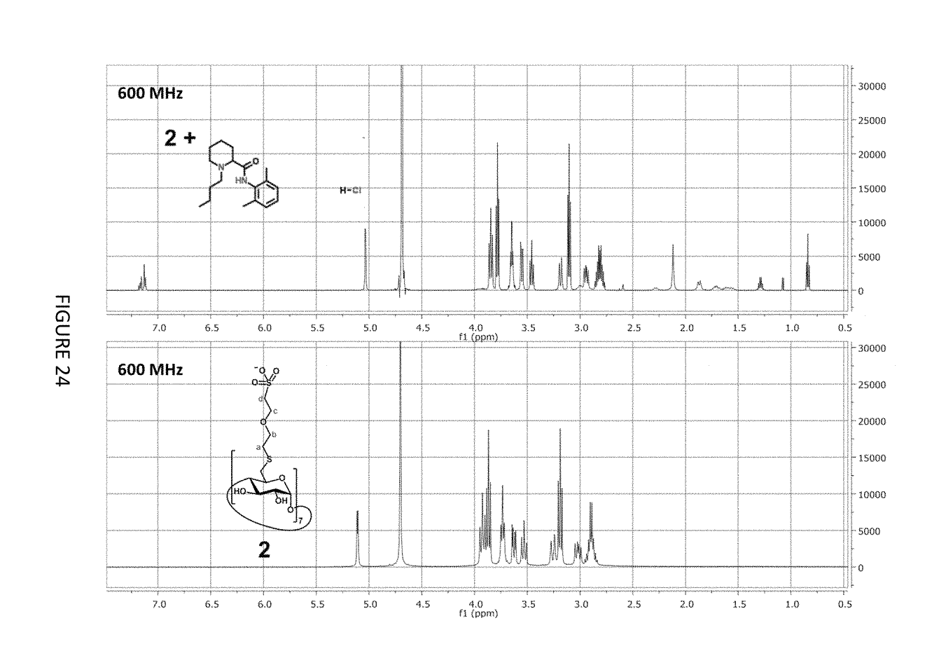

Bupivacaine hydrochloride is a local anaesthetic drug. FIG. 24 shows inclusion studies between the polyanionic beta-CD 2 and Bupivacaine hydrochloride by NMR (bottom panel: compound 2 alone, top panel: compound 2 with Bupivacaine hydrochloride). Significant changes in chemical shifts of host CD molecule (2) before and after mixing with Bupivacaine hydrochloride were observed, suggesting the polyanionic CD hosts in accordance with the present invention may be used to form inclusion complexes with Bupivacaine hydrochloride and analogs thereof.

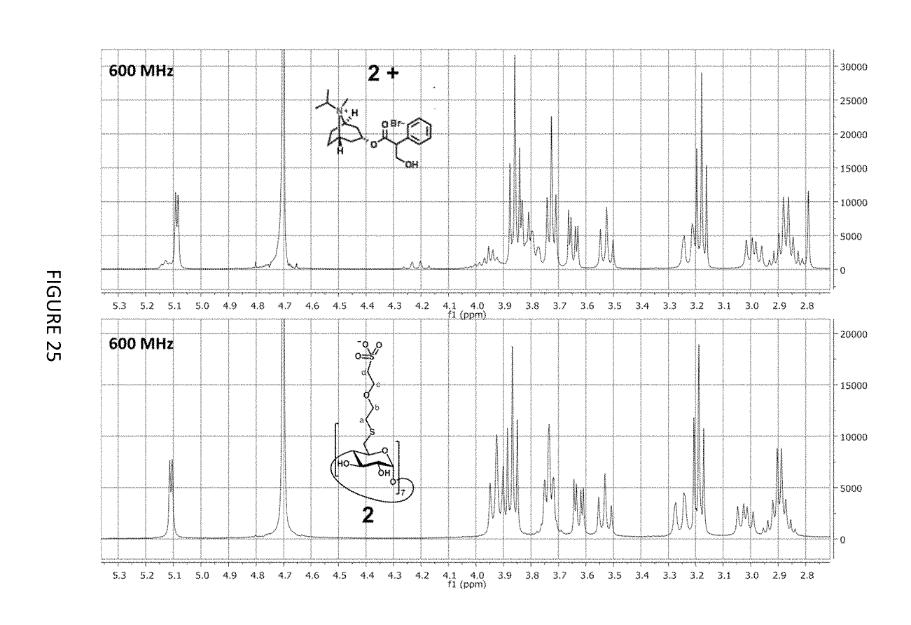

Ipratropium Bromide is an anticholinergic drug used for the treatment of chronic obstructive pulmonary disease and acute asthma. FIG. 25 shows inclusion studies between the polyanionic beta-CD 2 and Ipratropium Bromide by NMR (bottom panel: compound 2 alone, top panel: compound 2 with Ipratropium Bromide). Significant changes in chemical shifts of host CD molecule (2) before and after mixing with Ipratropium Bromide were observed, suggesting the polyanionic CD hosts in accordance with the present invention may be used to form inclusion complexes with Ipratropium Bromide and analogs thereof.

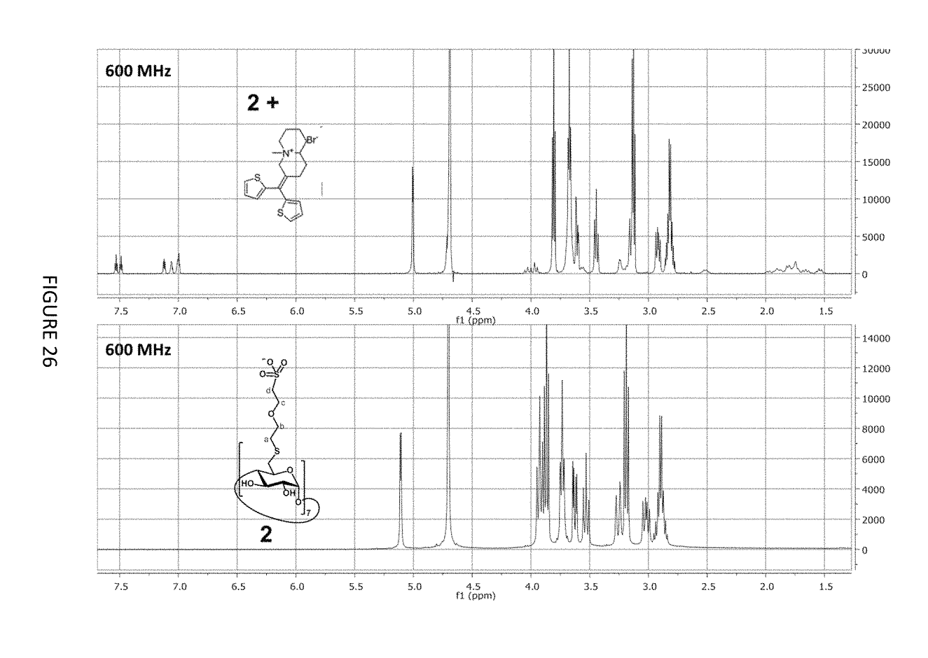

Tiquizium Bromide is an antimuscarinic agent used as an antispasdomdic pain mediating drug. FIG. 26 shows inclusion studies between the polyanionic beta-CD 2 and Tiquizium Bromide by NMR experiment (bottom panel: compound 2 alone, top panel: compound 2 with Tiquizium Bromide). Significant changes in chemical shifts of host CD molecule (2) before and after mixing with Tiquizium Bromide were observed, suggesting the polyanionic CD hosts in accordance with the present invention may be used to form inclusion complexes with Tiquizium Bromide and analogs thereof.

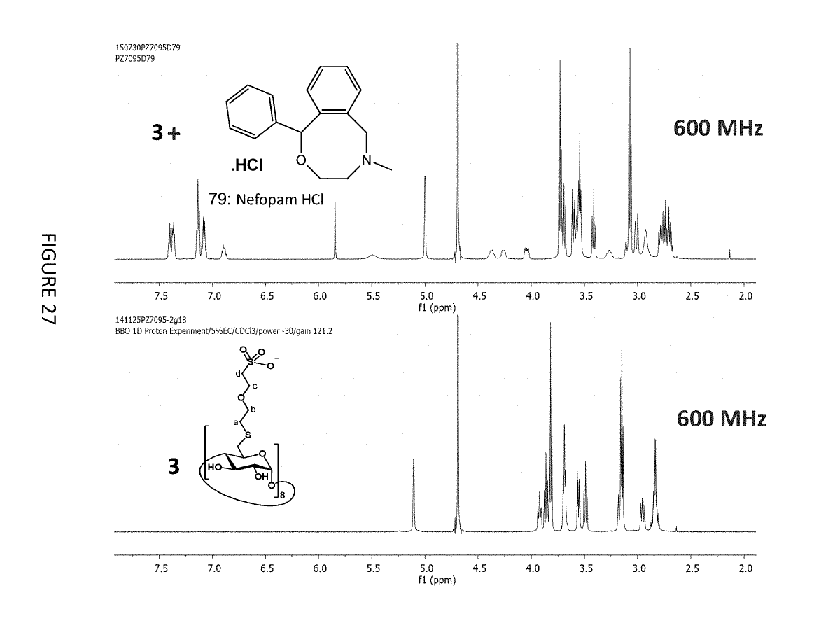

FIG. 27 illustrates NMR results for the inclusion of nefopam with structure 3.

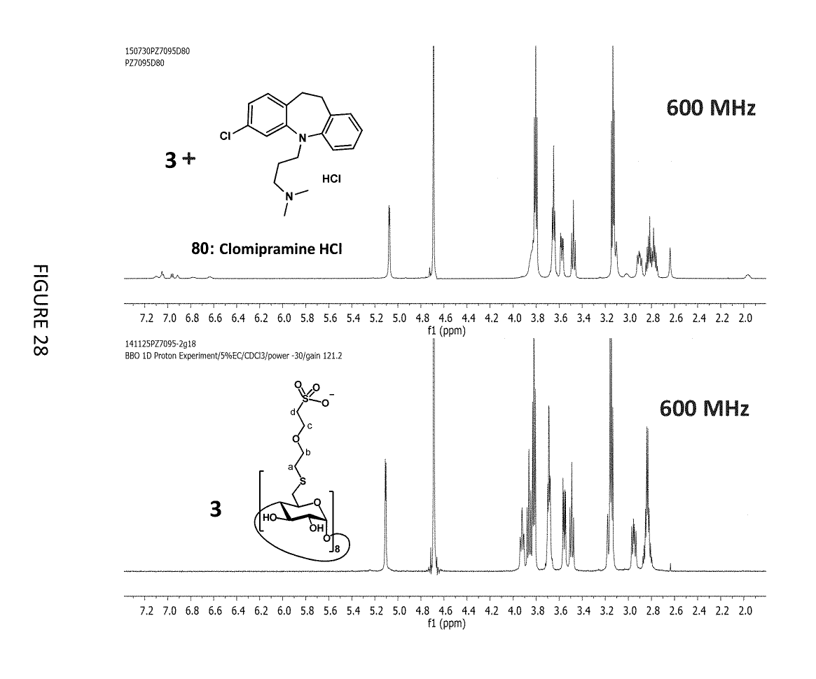

FIG. 28 illustrates NMR results for the inclusion of clomipramine with structure 3.

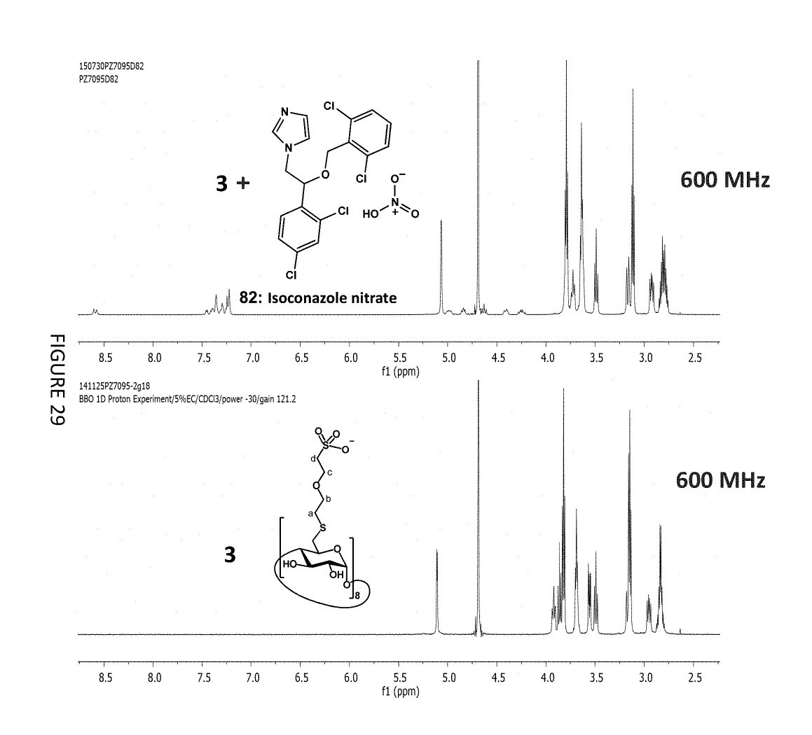

FIG. 29 illustrates NMR results for the inclusion of isoconazole nitrate with structure 3.

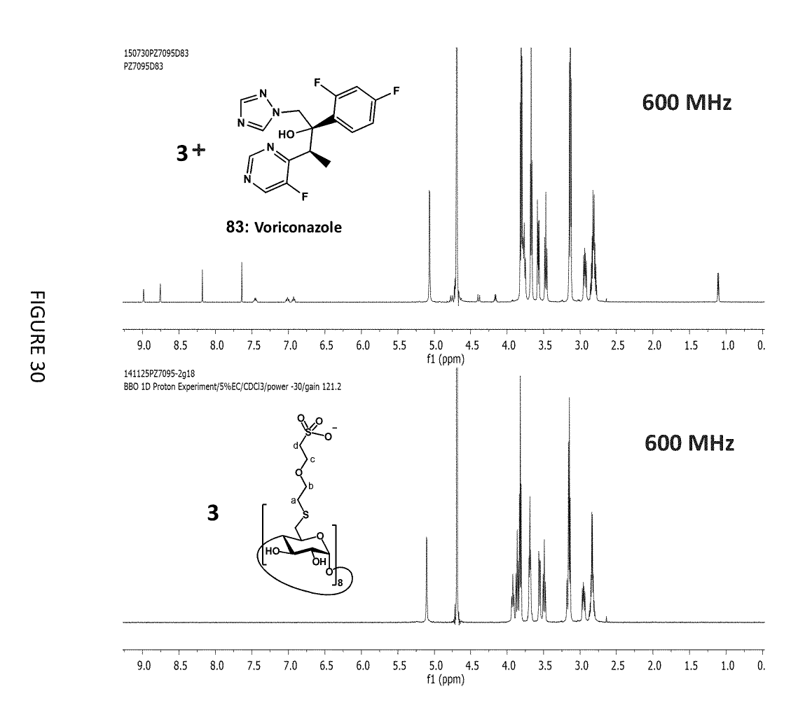

FIG. 30 illustrates NMR results for the inclusion of voriconazole with structure 3.

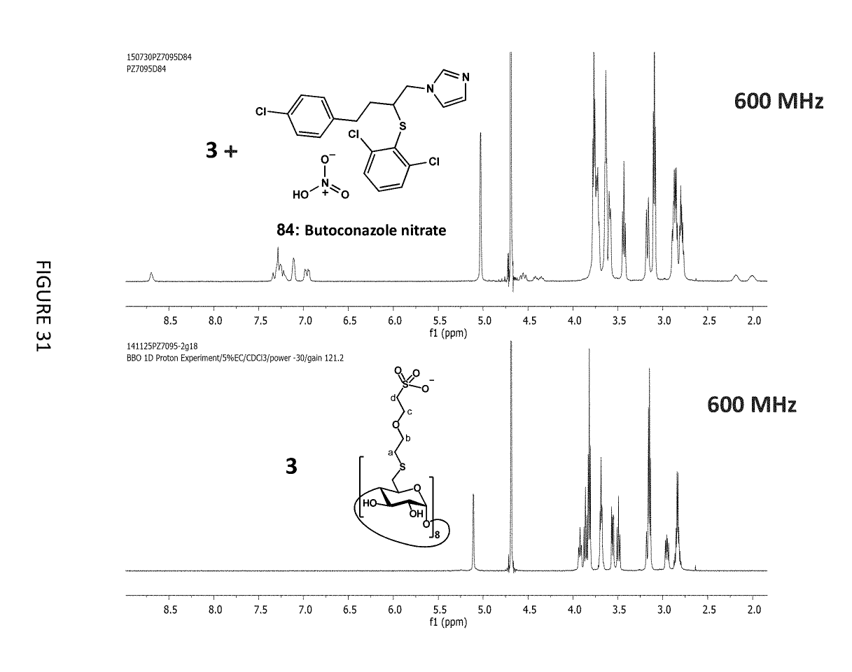

FIG. 31 illustrates NMR results for the inclusion of butoconazole nitrate with structure 3.

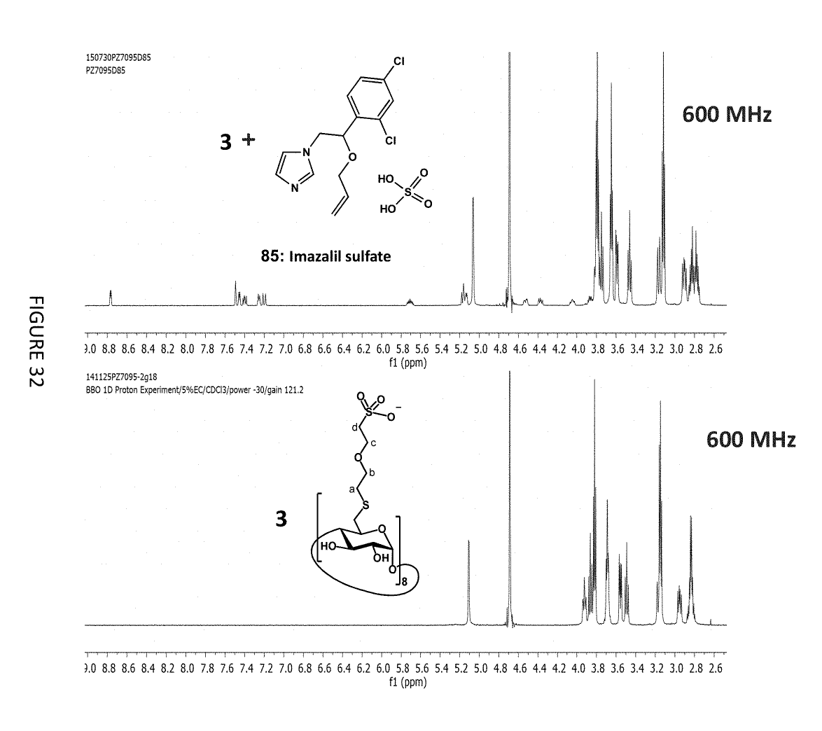

FIG. 32 illustrates NMR results for the inclusion of imazalil sulfate with structure 3.

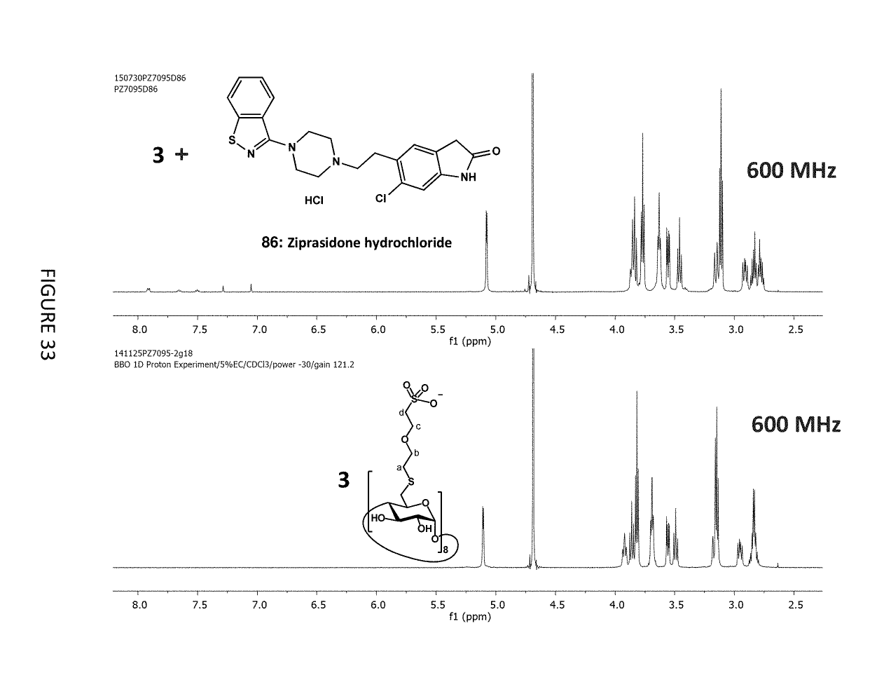

FIG. 33 illustrates NMR results for the inclusion of ziprasidone HCl with structure 3.

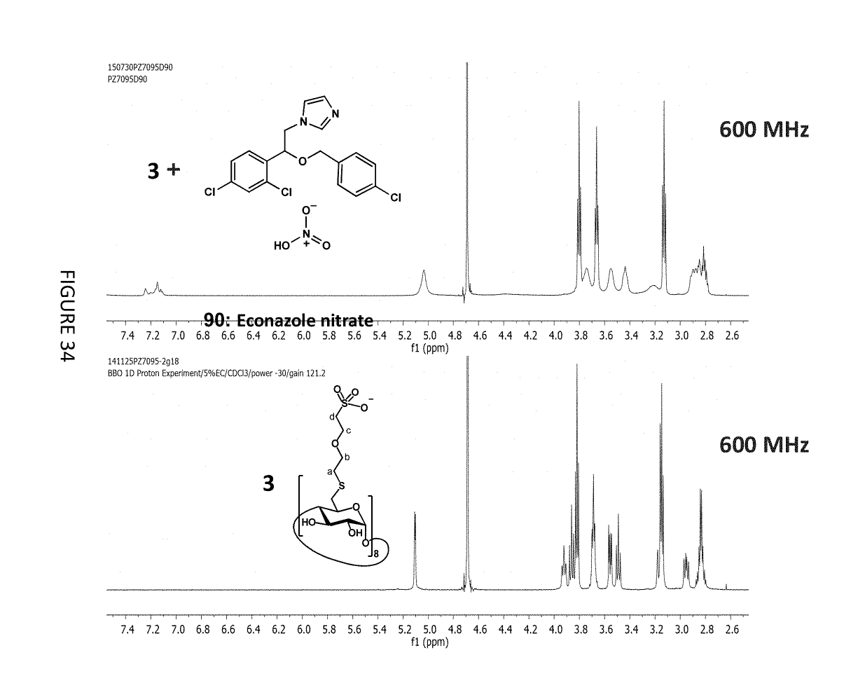

FIG. 34 illustrates NMR results for the inclusion of econazole nitrate with structure 3.

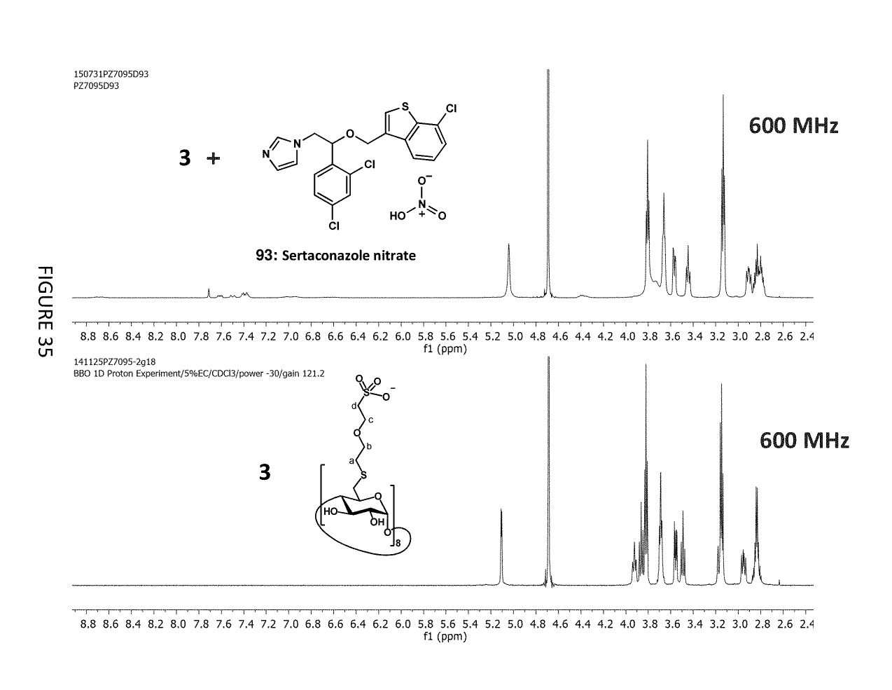

FIG. 35 illustrates NMR results for the inclusion of sertaconazole nitrate with structure 3.

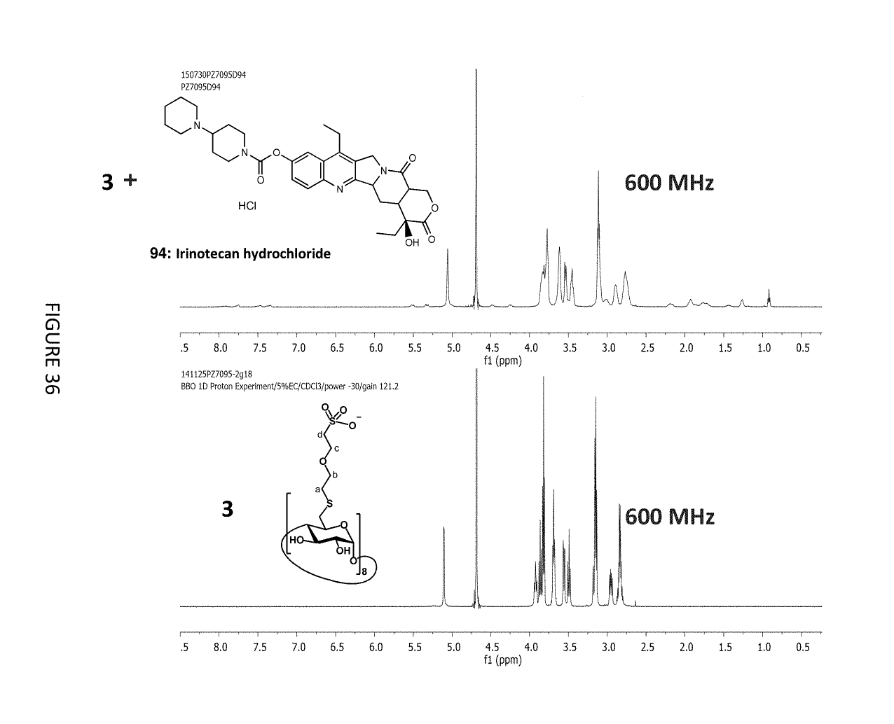

FIG. 36 illustrates NMR results for the inclusion of irinotecan HCl with structure 3.

### Example 5: Inclusion Studies with Commercial Medicines by Electrospray Mass Spectrometry and Binding Constant Determination

In this example, results from mass spectrometry are provided. These results illustrate the inclusion of various drugs with exemplary polyanionic cyclodextrin dendrimers

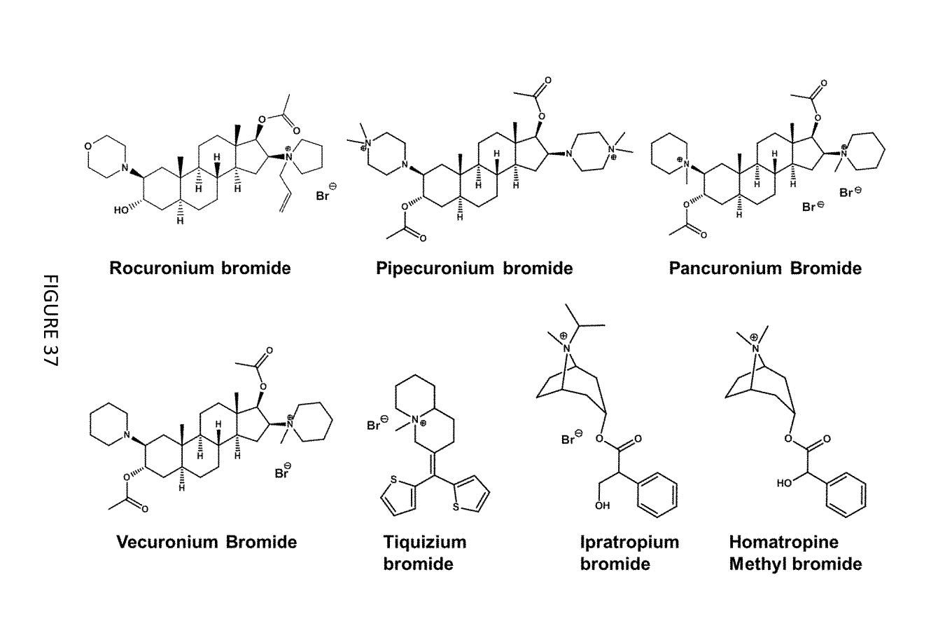

FIG. 37 shows structures of selected commercial medicines (Rocuronium bromide, Pipecuronium Bromide, Pancuronium Bromide, Vecuronium Bromide, Tiquizium Bromide, Ipratropium Bromide and Homatropine Methyl bromide) used to measure binding constants with both polyanionic gamma-CDs 3 and 6.

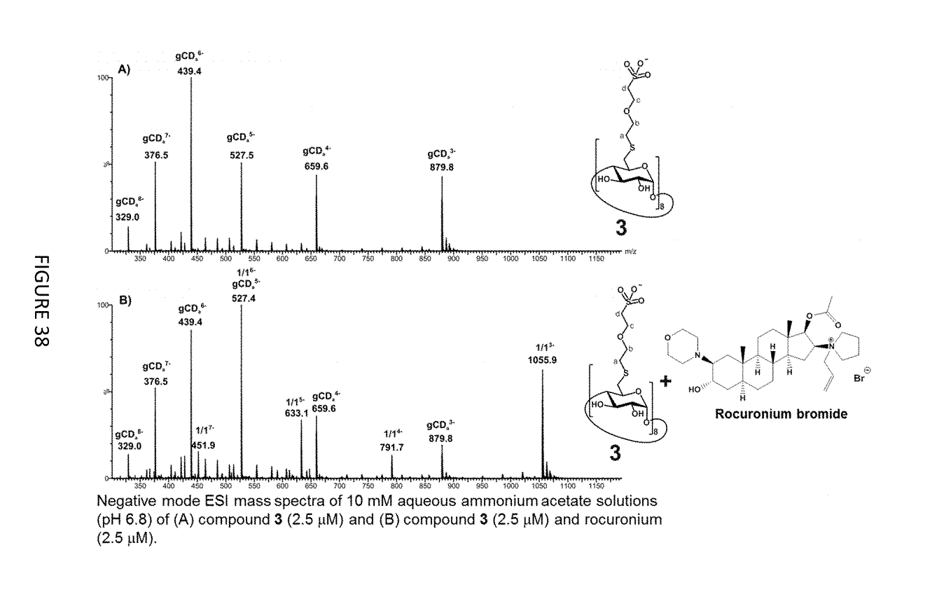

FIG. 38 shows an exemplary inclusion study of polyanionic sulfoPEG gamma-CD derivative 3 with rocuronium bromide by ESI-mass spectrometry (Top panel: compound 3 alone, bottom panel: compound 3 with Rocuronium Bromide).

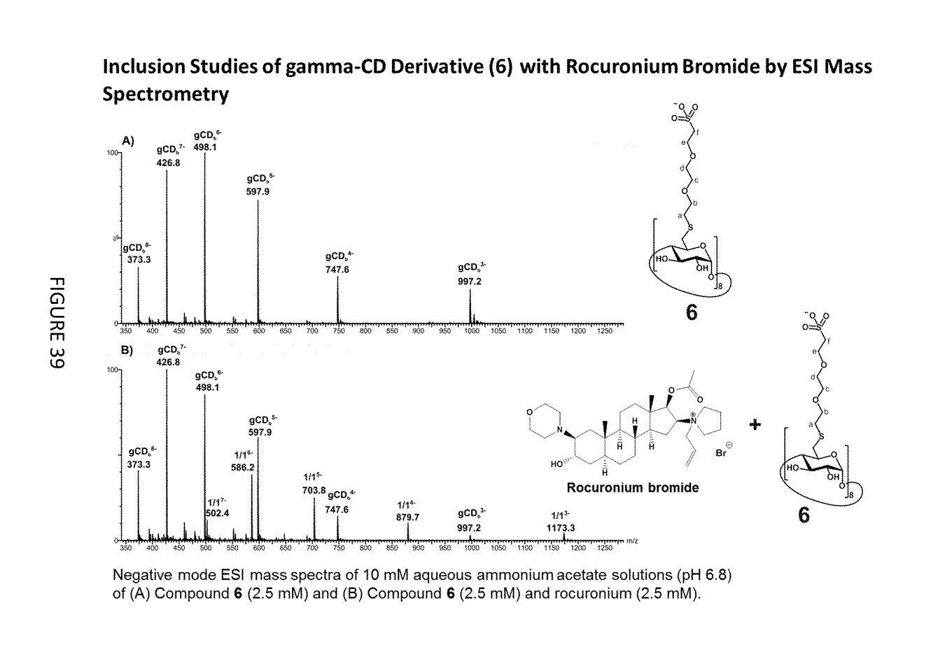

Analogously, FIG. 39 shows an exemplary inclusion study of polyanionic sulfoPEG gamma-CD derivative 6 with rocuronium bromide by ESI-mass spectrometry (Top panel: compound 6 alone, bottom panel: compound 6 with Rocuronium Bromide). The negative mode ESI mass spectra were obtained using 10 mM aqueous ammonium acetate solutions (pH 6.8) of CD host (either compound 3 or 6, 2.5 mM), and CD host (compound 3 or 6, 2.5 mM) combined with rocuronium bromide (2.5 mM). Characteristic m/z peaks corresponding to CD host at charge states −3 to −8, and to the (CD+drug) complexes at charge states −3 to −7 were observed.

The apparent association constant (Kd,app) for the (CD+drug) complexes were calculated from the ESI mass spectra using the equation:

$${K}_{d , app} = \frac{[ CD ] \times {[ drug ]}_{free}}{[ CD + drug ]} , \qquad ( 1 )$$

where [CD+drug]; [CD] and [drug]_{free} are the concentrations of complex, free CD and drug, respectively. The equilibrium concentrations were calculated from the relative abundances (Ab) of (CD+drug) and CD ions measured by ESI-MS and the mass balance considerations, using following equations:

$$\begin{matrix}[ CD ] = {[ CD ]}_{0} \times \frac{Ab ( CD )}{Ab ( CD ) + Ab ( CD + drug )} ; & ( 2 ) \\ [ CD + drug ] = {[ CD ]}_{0} \times \frac{Ab ( CD + drug )}{Ab ( CD ) + Ab ( CD + drug )} ; & ( 3 ) \\ {[ drug ]}_{free} = {[ drug ]}_{0} - [ CD + drug ] . & ( 4 )\end{matrix}$$

The ESI-MS measurements were performed at three different concentrations of CD and rocuronium and three replicate measurements were performed at each concentration. From these measurements, Kd,app values of 1.2 (±0.1)×10⁻⁶ M and 6.5 (±0.1)×10⁻⁶ M were determined for CD hosts 3 and 6, respectively (Table 1). Notably, these values are in excellent agreement with values measured using isothermal titration calorimetry (ITC), 1.5 (±0.2)×10⁻⁶ M and 7.7 (±0.9)×10⁻⁶ M, respectively, suggesting both polyanionic compounds 3 and 6 form very strong inclusion complexes with rocuronium bromide.

TABLE 1
K_{d,app} for CD (Compound 3 and 6) binding to rocuronium
bromide measured by ESI-MS and ITC at 25° C., in 10 mM
ammonium acetate, pH 6.8.

K_{d,app} (×10⁻⁶M)

| Complex | ESI-MS | ITC |
|---|---|---|
| Compound 3 | 1.2 (±0.1) | 1.5 (±0.2) |
| Compound 6 | 6.5 (±0.1) | 7.7 (±0.9) |

Having established that the ESI-MS measurements provide a reliable binding, the assay was used to quantify binding of CD hosts 3 and 6 to the other selected drug molecules listed in FIG. 37. Binding was detected for all of the cationic drug molecules, with K_{d,app} ranging from 1.2×10 to 1.1×10⁻³ M (Table 2). These data indicate that both polyanionic compounds 3 and 6 can bind to commercial medicines with different affinities.

TABLE 2
K_{d,app} for CD (Compound 3 and 6) binding to drugs (d2-d7) measured by
by ESI-MS in 10 mM ammonium acetate, pH 6.8.

| Complex | K_{d,app} (×10⁻⁶M) | |
|---|---|---|
| Ligand | Compound 3 | Compound 6 |
| Rocuronium bromide (d1) | 1.2 (±0.1) × 10⁻⁶ | 6.5 (±0.1) × 10⁻⁶ |
| Pipecuronium bromide (d2) | 2.2 (±0.1) × 10⁻⁶ | 1.8 (±0.1) × 10⁻⁶ |
| Vecuronium bromide (d3) | 5.6 (±0.2) × 10⁻⁶ | 3.1 (±0.7) × 10⁻⁵ |
| Pancuronium bromide (d4) | 8.0 (±0.6) × 10⁻⁶ | 3.5 (±0.2) × 10⁻⁵ |
| Tiquizium bromide (d5) | 5.4 (±0.2) × 10⁻⁴ | 1.7 (±0.1) × 10⁻⁴ |
| Homatropine methylbromide (d6) | 9.8 (±0.2) × 10⁻⁴ | 1.1 (±0.1) × 10⁻³ |
| Ipratropium bromide (d7) | 8.6 (±0.1) × 10⁻⁴ | 6.2 (±0.1) × 10⁻⁴ |

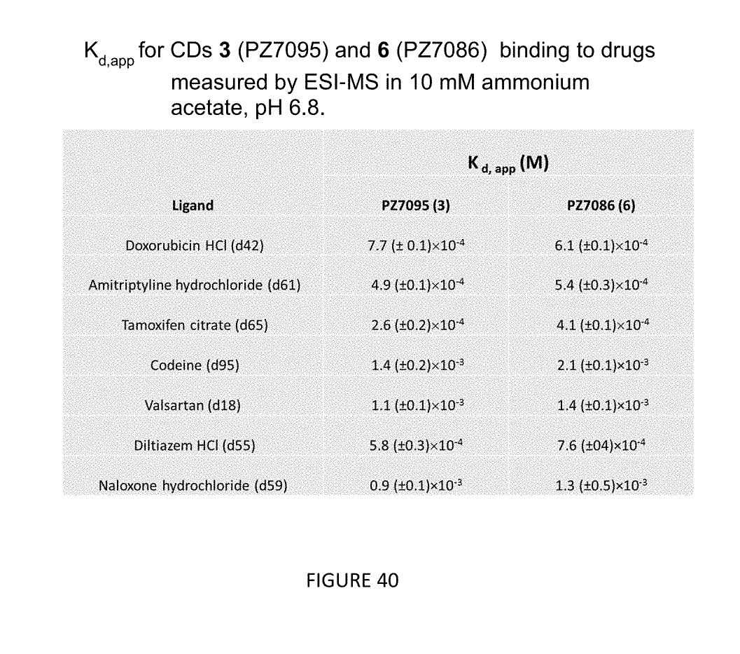

FIG. 40 shows Kd,app for CDs structure 3 (PZ7095) and structure 6 (PZ7086) binding to various drugs measured by ESI-MS in 10 mM ammonium acetate, pH 6.8.

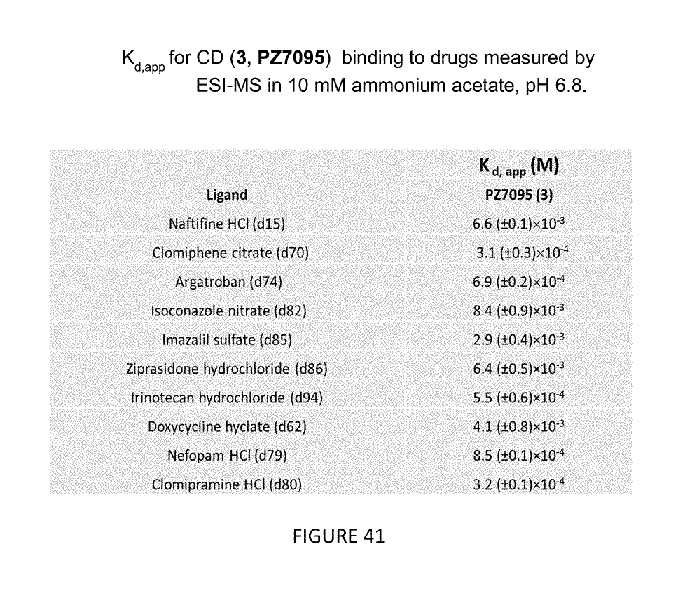

FIG. 41 shows Kd,app for CD (structure 3, PZ7095) binding to various drugs measured by ESI-MS in 10 mM ammonium acetate, pH 6.8.

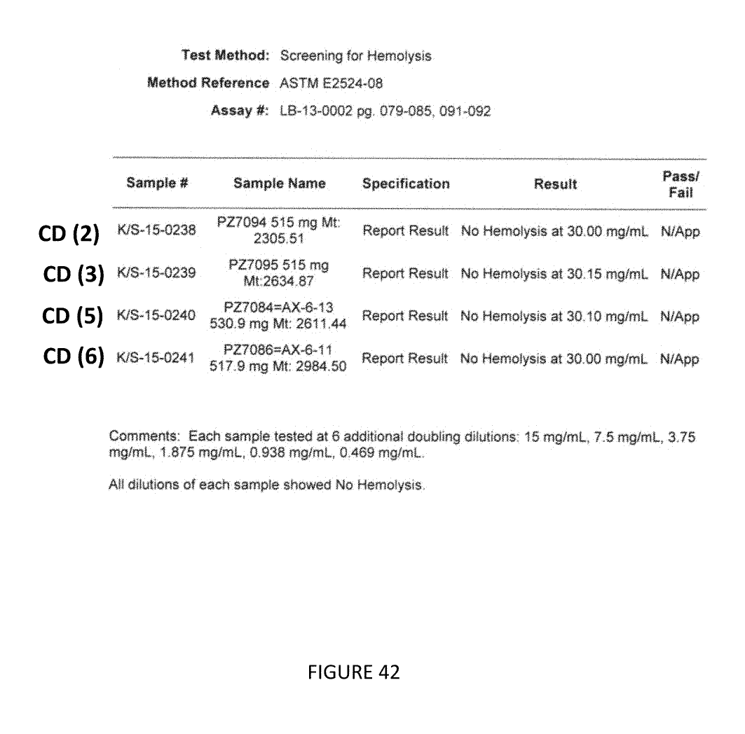

FIG. 42 shows hemolysis results for polysulfonate compounds 2-3, 5-6. Each sample was tested at 6 additional doubling dilutions: 15 mg/mL, 7.5 mg/mL, 3.75 mg/mL, 1.875 mg/mL, 0.938 mg/mL and 0.469 mg/mL. All dilutions of each sample showed no hemolysis.

### Example 6: A Maximum Tolerated Dose Toxicity Study of Compound 3 (PZ7095) Following Intravenous Injection in Sprague-Dawley Rats

Thirty individually housed Sprague-Dawley rats (15 males, 200-350 g; 15 females, 170-290 g) were divided into 5 groups (3 males and 3 females per group), and each animal received a single dose of compound 3 (PZ7095) according to the following dose levels: Group 1 (control): 0 mg/kg; Group 2: 100 mg/kg; Group 3: 350 mg/kg; Group 4: 1000 mg/Kg; Group 5: 3000 mg/kg. The injected volumes were 5 mL/kg for each animal.

All animals that survived were observed during a period of 8 days. At day 8, all animals were sacrificed. Prior to termination, blood and urine samples were collected for hematology, coagulation, clinical chemistry and urinalysis on individual animal.

During the In-Life phase, all animals were fed ad libitum, except for overnight food fast prior to blood collection for clinical chemistry analysis or necropsy, and the food consumption was recorded weekly. Water was provided ad libitum via water bottles.

Each animal was case-side observed twice daily for signs of mortality, moribundity, general health and signs of toxicity. Detailed clinical observations were also observed prior to dose on Day 1 and on the day of necropsy. These included changes in skin, fur, eyes, and mucous membranes, and also respiratory, circulatory, autonomic and central nervous system, and somatomotor activity and behavior pattern.

The body weight of individual animals was recorded prior to dose on Day 1, the day prior to necropsy and on the day of necropsy.

On Day 8, all surviving main study animals were euthanized and all animals were subjected to a full gross necropsy, which includes macroscopic examination of the external surface of the body, all orifices, cranial cavity, external surface of the brain, the thoracic, abdominal and pelvic cavities and their viscera, cervical areas, carcass and genitalia.

The organs of all scheduled-death animals were weighed as soon as possible at the scheduled necropsies. Paired organs will be weighed together.

Results

All animals in Groups 1-4 survived. For the six animals in Group 5 (the highest dose group), three male animals were found dead right after the dose, while all three female animals survived after the dose. Necropsy of the three dead male animals in Group 5 was performed according to protocols; however, no gross findings were observed.

All surviving animals from the dosing stayed alive until day 8. No gross findings were observed in all animal groups.

In conclusion, it appears that compound 3 (PZ7095) was well tolerated by Sprague-Dawley rats at single intravenous dose up to 1000 mg/Kg. The fact that three female rats of even higher dose group (Group 5) remained healthy may suggest that compound 3 (PZ7095) could be well tolerated at even higher dose level than 1000 mg/Kg.

### Example 7: Synthesis and Inclusion Studies of Exemplary Polycarboxylates with Various Drugs

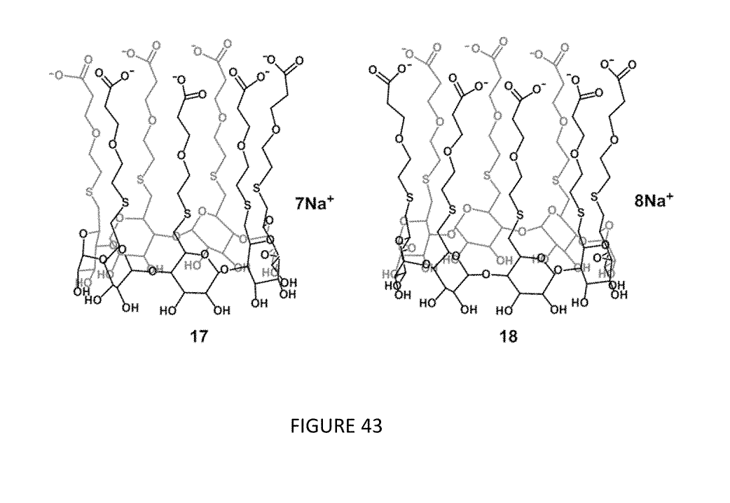

FIG. 43 shows two examples of a caroboxyPEG thioether in accordance with the present invention.

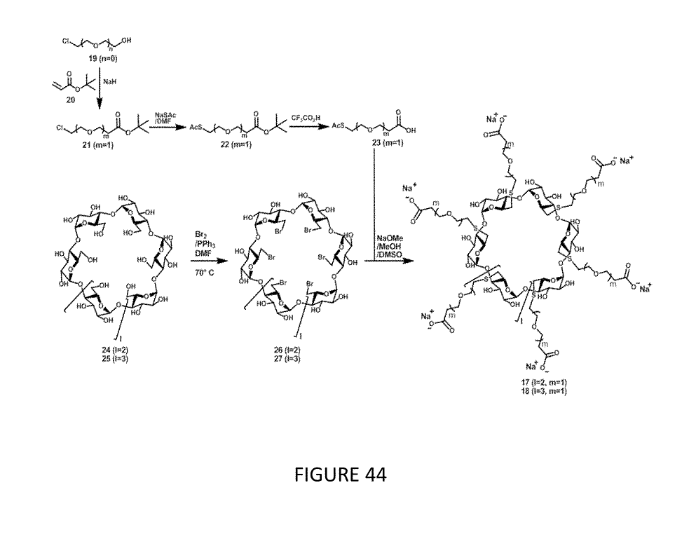

FIG. 44 shows an exemplary synthesis of these carboxyPEG thioether CD analogs from per-6-bromo-cyclodextrins. The required thioacetate containing a terminal carboxy group (23) was prepared from monochlorinated PEGs, by first carrying out a Michael addition to tert-butyl acrylate, followed by displacing the chloride with a thioacetate, and finally the ter-butyl group is smoothly removed with trifluoroacetic acid.

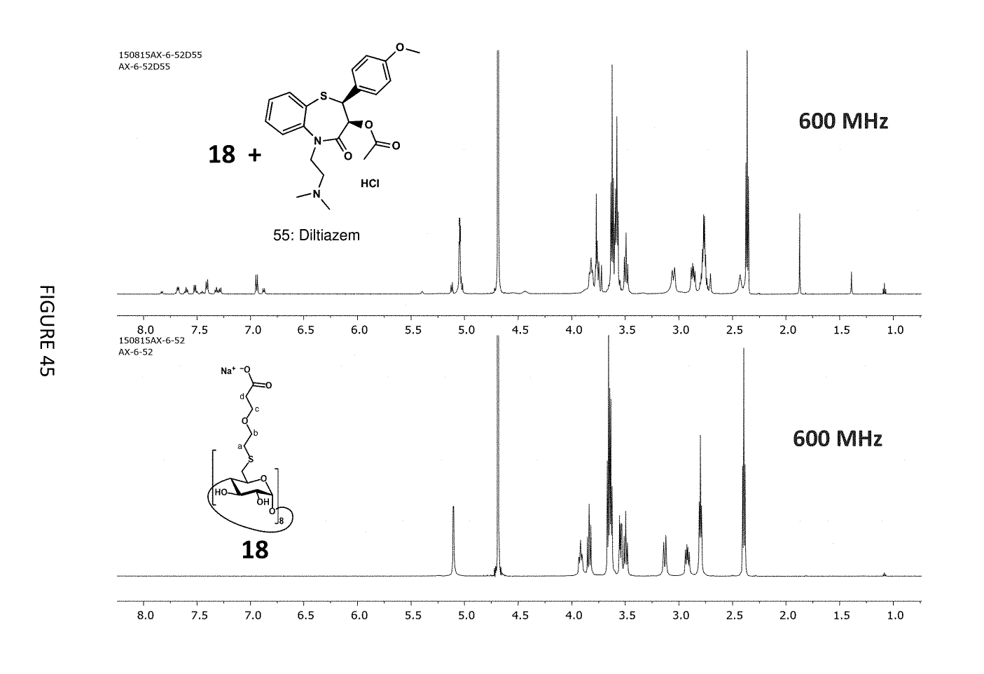

FIG. 45 illustrates ¹H NMR spectrum (bottom) of obtained polycarboxylate 18, which show high purity.

FIG. 45 also show results for the inclusion of diltiazem with structure 18.

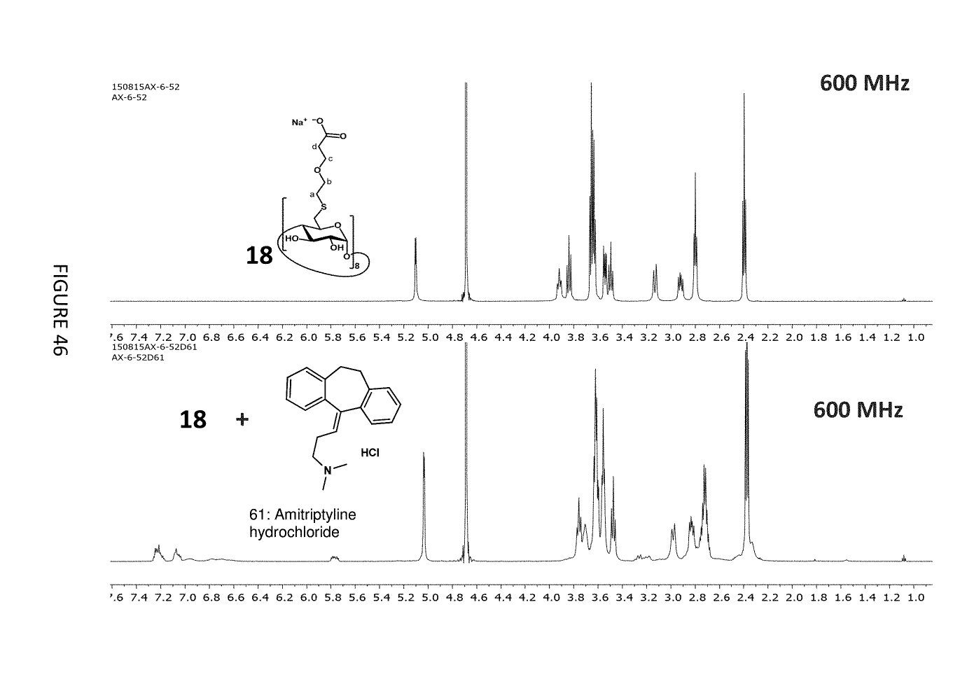

FIG. 46 illustrates NMR results for the inclusion of amitripline with structure 18.

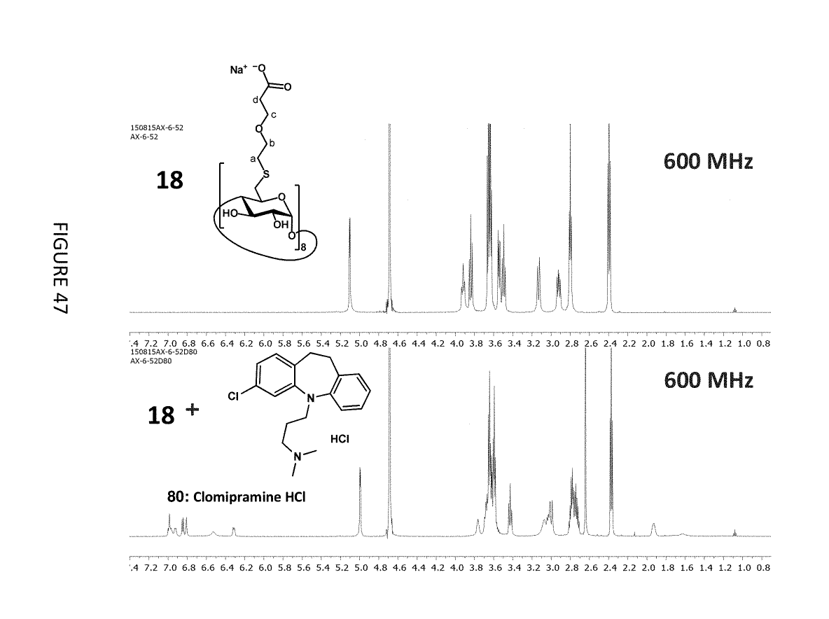

FIG. 47 illustrates NMR results for the inclusion of clomipramine with structure 18.

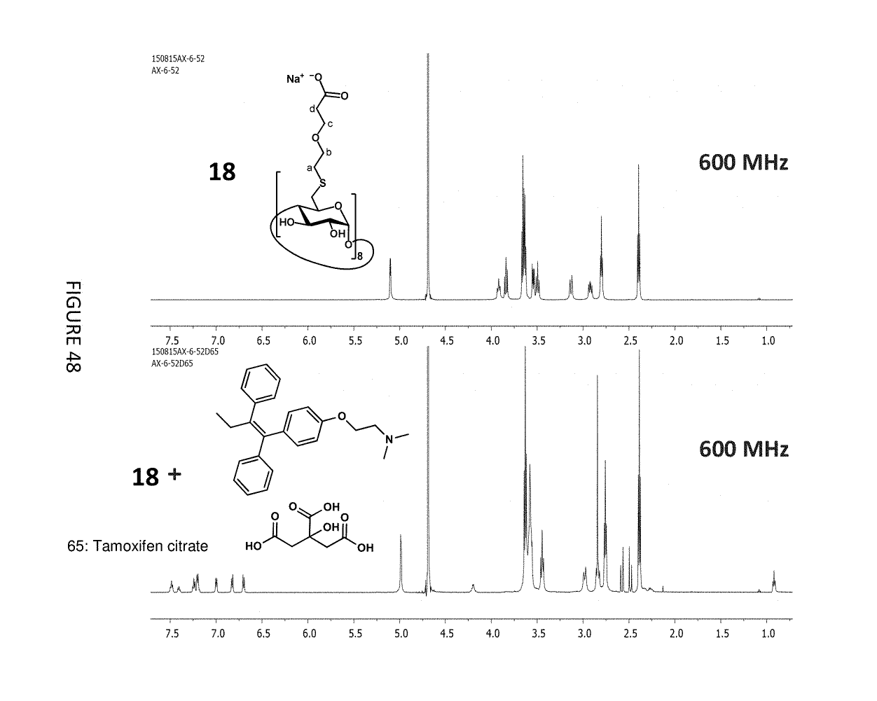

FIG. 48 illustrates NMR results for the inclusion of tamoxifen citrate with structure 18.

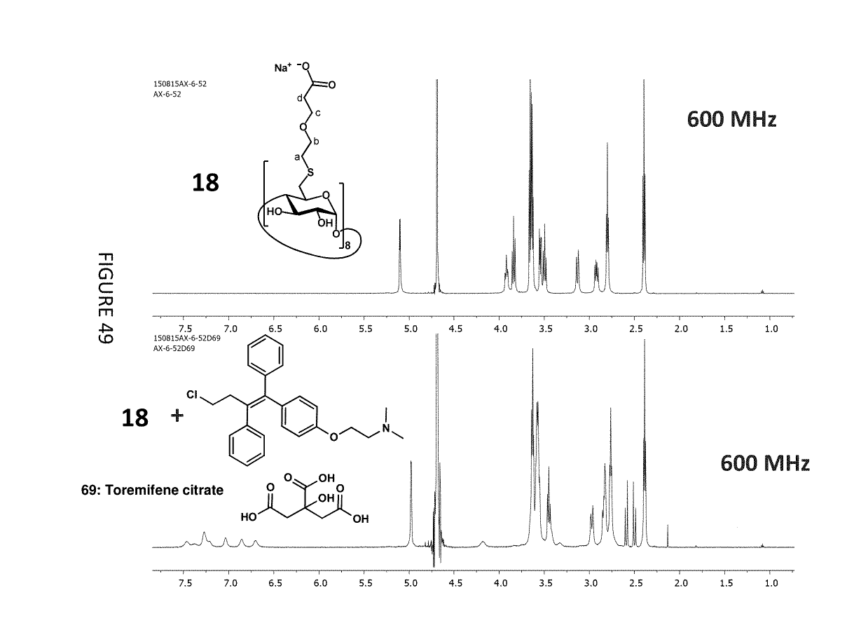

FIG. 49 illustrates NMR results for the inclusion of toremifene citrate with structure 18.

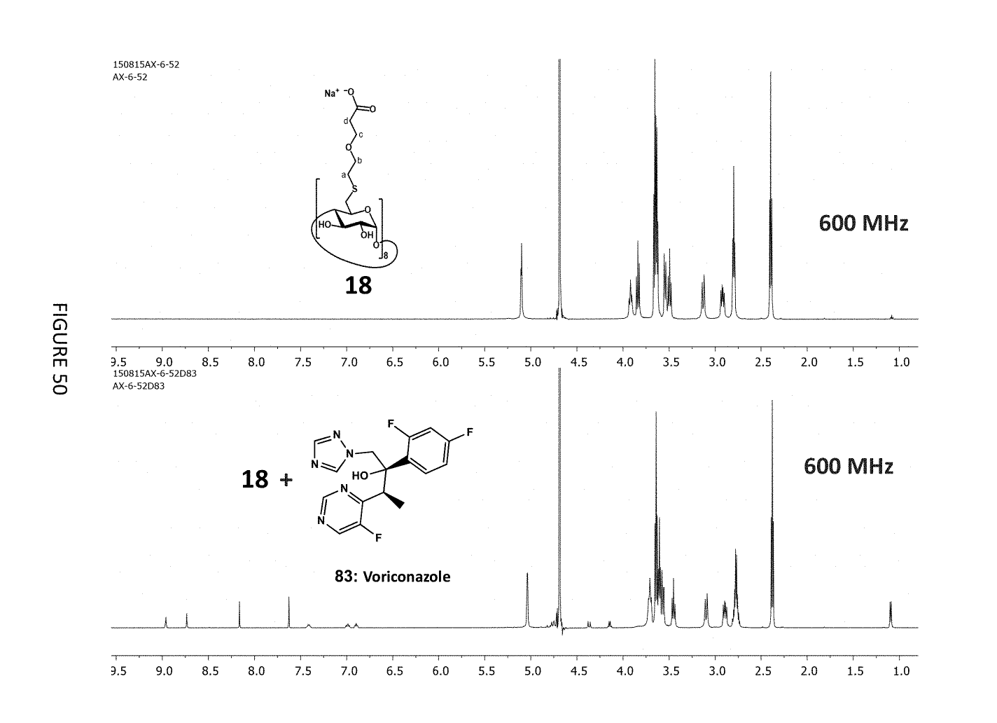

FIG. 50 illustrates NMR results for the inclusion of voriconazole with structure 18.

ESI-MS measurements were also performed on polycarboxylate structures 17 and 18 with rocuronium bromide. K_{d,app} values of 1.26(±0.10)×10⁻⁴ and 1.59(±0.10)×10⁻⁶ M were determined for CD hosts 17 and 18, respectively.

Thus, the above examples indicate that compounds as described herein may be used for inclusion of a variety of drugs, as excipients or rescue medicines.

### REFERENCES

U.S. Pat. No. 7,632,941, Defaye, J., et al., Cyclodextrin Derivatives, Method for the Preparation thereof and Use thereof for the Solubilization of Pharmacologically Active Substances.
U.S. Ser. No. 12/374,211, Defaye, J. (Centre National de la Recherche Scientifique), Novel Amphiphilic Cyclodextrin Derivatives.
PCT/FR2004/000691, Defaye, J. (Centre National de la Recherche Scientifique), Novel Cyclodextrin Derivatives, Methods for Preparation Thereof and use for the Solubilization of Pharmacologically Active Substances.
U.S. Pat. No. 6,670,340, 6-Mercapto-Cyclodextrin Derivatives: Reversal Agents for Drug-Induced Neuromuscular Block.
U.S. Pat. No. 6,949,527, 6-Mercapto-Cyclodextrin Derivatives: Reversal Agents for Drug-Induced Neuromuscular Block.
DE102010012281, Bichimaier, I., Pharmazeutische zusammensetzungen enthaltend substituiertes 6-deoxy-6-sulfanylcyclodextrin.
Bull. Chem. Soc. Chim. Fr. 132 (8), 857-866, 1995.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1-40. (canceled)

41. A pharmaceutical composition comprising a polyanionic compound of the formula: 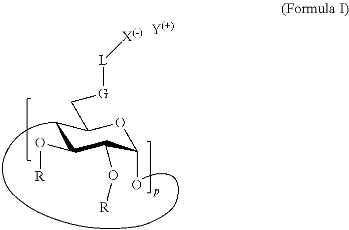
wherein
X⁽⁻⁾ is one or more negatively charged moieties,
Y⁽⁺⁾ is Na⁺ or K⁺;
L comprises a substituted or unsubstituted alkyl group, and/or a substituted or unsubstituted polyethylene glycol (PEG) group, or a combination of one or more alkyl groups and one or more PEG groups;
p is 6, 7, or 8;
G is a bond or represents any one or more suitable bridging groups, or a substituted or unsubstituted 1,2,3-triazole;
R is H, an optionally substituted alkyl group or an optionally substituted acyl group; and
together with a pharmaceutically acceptable diluent.

42. The composition of claim 41, wherein X⁽⁻⁾ is —SO₃⁻, —CO₂⁻, —OSO₃⁻, or —OPO₃⁻.

43. The composition of claim 41, wherein L is a C₁-C₁₁ alkyl group.

44. The composition of claim 41, wherein p is 7.

45. The composition of claim 41, wherein G is an ester, amide, amine, or sulfur or comprises a group substituted with a group comprising one of the following structures:

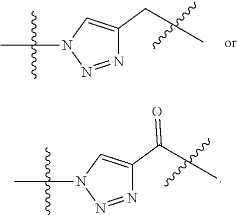 [SMILES: *Cc1cn(*)nn1 ; *C(=O)c1cn(*)nn1]

46. The composition of claim 45, wherein G is —S—, —OC(O)—, —NHC(O)—, —SO—, or —SO₂—.

47. The composition of claim 41, wherein R is an optionally substituted C₁-C₁₈ alkyl group or acyl group.

48. The composition of claim 41, wherein the PEG group is of the formula —CHZ(CH₂OCHZ)ₘCH₂ where Z is H or CH₃ and m is 1 to 20.

49. The composition of claim 41, wherein L comprises: any unsubstituted or substituted alkyl group; an unsubstituted or substituted PEG group; or L comprises a PEG group which has none, or one or more alkyl groups flanking on either or both sides of the PEG group.

50. The composition of claim 49, wherein the alkyl group is substituted with a PEG group.

51. The composition of claim 49, wherein the PEG group is substituted with one or more alkyl groups.

52. The composition of claim 41, wherein one or more of the CH₂ groups of the alkyl groups is replaced with an atom or functional group.

53. The composition of claim 52, wherein atom or functional group is —O—, —S—, —SO—, —SO₂—, —CONH—, —COO—, —NZ—, or a substituted or unsubstituted 1,2,3-triazole group.

54. The composition of claim 53, wherein the 1,2,3-triazole group is substituted with a group comprising one of the following structures:

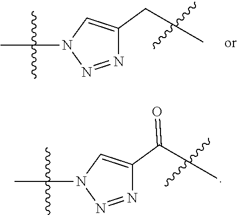 [SMILES: *Cc1cn(*)nn1 ; *C(=O)c1cn(*)nn1]

55. A pharmaceutical composition comprising a non-ionic cyclodextrin-based compound of the formula: 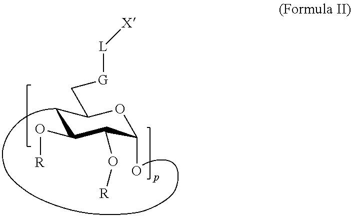
wherein
X′ is one or more neutral moieties,
L is one or more linkers,
G is a bond or is one or more bridging groups,
p is an integer, and
R is one or more substituents,
together with a pharmaceutically acceptable diluent.

56. A polyanionic cyclodextrin-based compound of the formula: 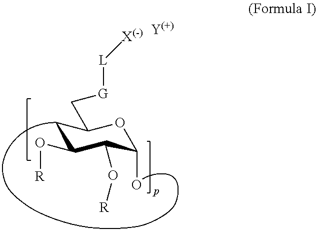
wherein
p is 6 (α-cyclodextrin), 7 (β-cyclodextrin) or 8 (γ-cyclodextrin),
X⁽⁻⁾ is —CO2 or —SO3-;
G is —S—;
L is —(CH₂)ₖ—, where k is 1 to 11, optionally 7 to 11; or L is 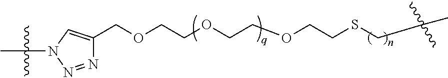
where q is 0 to 20 and n is 1-5, optionally 1-11, or 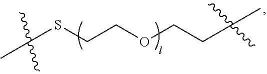
where 1 is 1-20; and
R is H, optionally substituted C₁-C₁₈ alkyl, or optionally substituted C₁-C₁₈ acyl.

57. A composition comprising the compound of claim 56, together with a pharmaceutically acceptable diluent.

58. A method of treating a subject in need thereof for removal of an undesired molecule in the subject, comprising administering the composition of claim 41, to said subject, such that the compound binds to said molecule, and removes it from said subject.

59. A method of treating a subject in need thereof for removal of an undesired molecule in the subject, comprising administering the composition of claim 56, to said subject, such that the compound binds to said molecule, and removes it from said subject.
